(19) United States
(12) Patent Application Publication
(10) Pub. No.: US 2005/0175541 A1
(43) Pub. Date: Aug. 11, 2005

(54) Enhanced drug delivery

(76) Inventors: Gregory M. Lanza, St. Louis, MO (US); Samuel A. Wickline, St. Louis, MO (US)

Correspondence Address: MORRISON & FOERSTER LLP, 3811 VALLEY CENTRE DRIVE, SUITE 500, SAN DIEGO, CA, 92130-2332, US

(21) Appl. No.: 10/993,578

(22) Filed: Nov. 19, 2004

Related U.S. Application Data
(60) Provisional application No. 60/523,833, filed on Nov. 19, 2003.

(51) Int. Cl. A61K 49/00; A61K 9/127

(52) U.S. Cl. 424/9.5; 424/450

(57) ABSTRACT

The invention is directed to methods for the delivery of a therapeutic agent using lipid-encapsulated particles containing the therapeutic agent and ultrasound energy. For example, it particularly relates to the use of ultrasound with a lipid-encapsulated emulsion comprising an oil where the emulsion is coupled a targeting ligand and comprises a therapeutic agent.

### CROSS-REFERENCE TO RELATED APPLICATIONS

This application claims benefit under 35 U.S.C. § 119(e) to provisional application 60/523,833 filed 19 Nov. 2003, which is herein incorporated by reference in its entirety.

### TECHNICAL FIELD

This invention relates generally to methods to enhance the delivery of a therapeutic agent using lipid-encapsulated particles containing the therapeutic agent and applying ultrasound energy in a manner wherein the lipid encapsulation is not disrupted. It particularly relates to the use of ultrasound with a lipid-encapsulated nanoparticle emulsion comprising an oil or perfluorocarbon where the emulsion is coupled a targeting ligand and comprises a therapeutic agent.

### BACKGROUND OF THE INVENTION

Ligand-targeted emulsions that include therapeutic agents on and/or in the emulsion are effective at delivering the agent to a particular target cell, organ or tissue. For example, liquid perfluorocarbon (PFC) nanoparticles have been used to deliver therapeutic agents to cells selectively by binding to specific cellular epitopes (Lanza et al. (2002) Circulation 106:2842-2847). In this case, a lipid/surfactant-wrapped, liquid perfluorocarbon (e.g., perfluorooctyl bromide, PFOB) emulsion was used to deliver the agent. Such PFC nanoparticles are distinct from conventional microbubble ultrasound contrast systems. The particles are targeted by incorporation of selected ligands (e.g., monoclonal antibodies, small molecules, etc.) into the lipid membrane through, for example, bifunctional intermediaries complexed to lipid adducts that situate within the lipid membrane of the particle. The perfluorocarbon nanoparticles also serve as an acoustic contrast agent by markedly enhancing reflectivity of surfaces to which they are bound by a mechanism entirely distinct from that of microbubbles. Lanza et al. (1998) J. Acoust. Soc. Am. 104:3665-3672.

Ultrasonic methods using microbubble agents (e.g., cavitation) have been used in attempts to enhance delivery of drugs, genes, and other therapeutic agents both in vitro and in vivo. See, for example, Dijkmans et al. (2004) Eur. J. Echocardiogr. 5:245-256; Guzman et al. (2001) J. Acoust. Soc. Am. 110:588-596; Liu et al. (1998) Pharmaceutical Res. 15:918-924; Postema et al. (2004) Ultrasound Med. Biol. 30:827-840; Song et al. (2001) J. Am. College of Cardiol. 39:726-731; Taniyama et al. (2002) Circulation 105:1233-1239. Cavitation, however, can potentially lead to cell and tissue destruction.

There remains a continuing need for development of methods and compositions that are useful for reaching a variety and/or particular sites and tissues within an individual and that result in an enhanced degree of specificity and therapeutic agent delivery. In particular, there remains a need for such methods and compositions that do not rely on cavitational mechanisms that may damage normal tissues and cells.

All publications and patent applications cited herein are hereby incorporated by reference in their entirety.

### DISCLOSURE OF THE INVENTION

The invention is directed to methods and compositions for improved delivery of therapeutic agents to targeted cells and/or tissue. The method comprises subjecting a nongaseous, lipid-encapsulated particle comprising a therapeutic agent to ultrasound energy at a frequency and mechanical index that enhances delivery of the agent to the target, wherein the particle is located at the target. During the application of ultrasound energy, the particle remains in a sufficiently non-gaseous state that the lipid encapsulation layer is not disrupted. Thus, the therapeutic agent is delivered without destruction of the particle itself and without creating temporary or permanent pores in cells (as in “sonoporation”) consequent to particle activation or destruction. The particle may be coupled to a targeting ligand to facilitate locating the particle at the target.

In applying the invention method, a suitable therapeutic agent is selected for delivery to an intended target, based on the diagnosis of the condition of the subject, or, if an in vitro method is employed, the nature of the modulation of cellular metabolism desired. The selected therapeutic and the targeting agent are designed to provide a specific treatment or prophylactic agent to a particular location. Thus, in one embodiment, the invention is directed to a method of treating a subject for a diagnosed disease or condition which method comprises selecting a therapeutic appropriate for said disease or condition and delivering that selected therapeutic by including it in non-gaseous lipid encapsulated particles, delivering the particles to the subject, and applying ultrasound energy as described above without disrupting the particles prior to drug delivery and without creating any artificial pores in cell membranes as is the case in traditional sonoporation.

### BRIEF DESCRIPTION OF THE DRAWINGS

FIG. 1A depicts an in vitro setup consisting of a phased array transducer, inverted microscope, and custom specimen holder, which permits ultrasound application with simultaneous visualization of cellular interaction.

FIG. 1B is an image at 2 MHz, 1.9 mechanical index (MI) of particles aligned perpendicular to direction of ultrasound propagation (see arrow) as a result of radiation forces.

FIG. 2 is a bar graph depicting the perfluorocarbon content associated with C32 melanoma cells for control or αᵥβ₃ targeted nanoparticles under normal and ultrasonically augmented conditions (n=12, +/−SEM, *p=0.01, **p=0.003, ANOVA).

FIG. 3 (top) is an image of fluorescein-labeled nanoparticles targeted to αᵥβ₃ integrins on C32 cells without ultrasound activation. The cell membrane staining indicates that mild lipid delivery has occurred. FIG. 3 (bottom) is an image of fluorescein-labeled nanoparticles targeted to αᵥβ₃ integrins on C32 cells with ultrasound activation. Note the marked augmentation of lipid delivery with ultrasound activation.

### MODES OF CARRYING OUT THE INVENTION

According to the present invention, nondestructive (i.e., noncavitational) ultrasound energy is used to enhance the interaction of nongaseous, lipid-encapsulated particles, including nanoparticles, with cell membranes and elicit enhanced therapeutic agent delivery without causing potentially harmful effects to other cells. Methods and compositions of the invention are of use in enhancing noncavitational therapeutic agent delivery. As illustrated herein, delivery of targeted perfluorocarbon (PFC)-based nanoparticles was enhanced using clinical levels of ultrasound energy.

Without being bound by a particular theory, the methods of the invention may use “radiation forces,” both primary and secondary, that are induced by traveling compressional waves, which can influence the particles by increasing contact with the targeted cell surface. The increased contact thereby facilitates improved transport of therapeutic compounds to the cell. Such forces may also improve particle binding to the targeted ligand by increasing contact with molecular epitopes (Dayton et al. (1999) Ultrasound in Medicine and Biology 25:1195-1201). The targeting ligand can also enhance the process by tethering the particle to the cell surface for prolonged ultrasound interaction.

This is in contrast to prior art methods which employ particulate delivery systems containing gas bubbles where the release of the drug is facilitated by effecting the disruption of the gas-containing particles by externally applied energy, such as ultrasound or by sonoporation methods which effect drug delivery through permanent or temporary membrane pores. The particles of the invention are non-gaseous when delivered to target sites and remain non-gaseous during the drug delivery process. (As explained below, minor amounts of gas may be present, but these are insufficient to effect disruption of the particles. Instead, the delivery of a therapeutic agent is effected by enhancing the interaction between the lipids encapsulating the delivery vehicles and the tissue itself, which may lead to direct fusion of the particle with the cell membrane or more simply lipid exchange.)

Lipid-encapsulated particles for use in the invention are modified to incorporate therapeutic agents including, but not limited to, bioactive, radioactive, chemotherapeutic and/or genetic agents, for use as a therapeutic agent and/or a diagnostic agent. The therapeutic agents may be on or attached at the surface of the lipid-encapsulated particles or within the core of the particles.

In some embodiments, the lipid-encapsulated particles can also serve as contrast agents and their delivery to the target can be detected by ultrasound imaging. Such particles would permit, for example, the site to be imaged in order to monitor the progress of the therapy on the site, to make desired adjustments in the dosage or therapeutic agent subsequently directed to the site and to make adjustments to the ultrasound energy directed to the particles.

As described herein, lipid-encapsulated particles appropriate for use in the present invention are nongaseous particles which include, but are not limited to, lipid-encapsulated nanoparticles, lipid-encapsulated liposomes, lipid-encapsulated emulsions, and lipid-encapsulated micelles.

In some embodiments, the invention provides use of the nongaseous, lipid-encapsulated particles for preparing a medicament for improving delivery of a therapeutic agent to a target upon the use of ultrasound energy after administration and localization of the particles at the target. The medicament may be for use in prophylactic measures or in treating a subject diagnosed with a disease or condition.

The invention provides methods of using the particles in a variety of applications including in vivo, ex vivo, in situ and in vitro applications.

The use of ultrasound energy with targeted particles incorporating at least one therapeutic agent is particularly useful for the treatment of a disease or disorder that has improved risk/benefit profiles when applied specifically to selected cells, tissues and/or organs. Application of ultrasound pulses to site-directed, lipid-encapsulated particles at an effective frequency and mechanical index provides delivery of therapeutic agents with enhanced efficiency to targeted tissues while decreasing potentially harmful effects to non-targeted cells associated with other forms of drug delivery. Without being bound to one particular theory, ultrasonically-enhanced delivery of therapeutic agents provides a noncavitational delivery mechanism unrelated to traditional sonoporation, or the formation of small temporary or even permanent pores in cell membranes induced by ultrasonic forces in concert with gaseous contrast agents. Accordingly, methods of the invention are useful in augmenting therapeutic agent delivery to a particular cell or tissue while limiting undesirable effects on non-targeted cells or tissues.

Methods of Enhancing Therapeutic Agent Delivery

The invention provides methods for improved delivery of therapeutic agents to targeted cells and/or tissue. The methods comprise pulsing ultrasound energy to a nongaseous, lipid-encapsulated particle comprising a therapeutic agent where the particle is located at the target. The ultrasound energy is provided at a frequency and mechanical index that enhances delivery of the agent to the target as compared to delivery of the agent from the use of the particle alone. The increased frequency and/or duration of lipid surface interactions between the target cell and the particle as a result of the ultrasound pulsing substantially enhances the net transfer of the agent to the target cell membrane or target cell beyond the effect of diffusion alone. At sufficient levels of acoustic pressure from the ultrasound, the particles at the target could be merged with the target cell and incorporated into the cell by lipid vesicle fusional processes.

The lipid-encapsulated particle comprising the therapeutic agent may or may not further comprise a targeting ligand. In some embodiments, the particle is coupled to a targeting ligand and, thus, directed to the target.

The use of ultrasound with the targeted, lipid-encapsulated particles containing a therapeutic agent provides enhanced delivery of the agent to the targeted cell both in in vitro and in vivo settings. It is also possible to deliver imaging agents to other cells such as lipid-conjugated compounds containing lanthanides (e.g., gadolinium), radionuclides, iron oxides, optically active agents (e.g., fluorophores), or x-ray contrast agents (e.g., iodine), among others.

During the application of ultrasound energy, the particle remains in a sufficiently non-gaseous state that the lipid encapsulation layer of the particle is not disrupted. As used herein, “disruption” of the lipid-encapsulated particle or lipid-encapsulation layer of the particle refers to something other than fusion of the lipid coating with the cell membrane, i.e. “disruption” refers to bursting the particle or creating pores in the encapsulation lipid. Thus, “disruption” does not include lipid exchange between the particle and cell membrane or direct fusion of the particle with the cell membrane.

For the methods of the invention, phased array transducers are typically used but single element transducers may also be used. The ultrasound energy delivered depends on frequency, mechanical index and time of exposure. In the methods, focused ultrasound energy is typically provided at clinical frequencies and powers. The frequency and mechanical index of the ultrasound and time of exposure can all be adjusted to optimize agent delivery by one skilled in the art. Mechanical indexes for use in the methods are at therapeutically reasonable levels or higher. For example, in some instances, mechanical indexes of about 1.0 to about 1.9 may be used and in other instances, mechanical indexes of about 0.5 to about 1.0 may be used. In some instances, mechanical indexes greater than 1.9 may be used. In certain cases, lower mechanical indexes of about 0.1 to about 0.5 may be used for longer periods of time to effect drug transfer. The ultrasound energy may be delivered using existing pulsing sequences and these pulsing frequencies may be optimized or specialized pulsing sequences may be developed to enhance lipid exchange.

In some embodiments, targeted cells can also be identified using ultrasound imaging techniques, for example, and agent delivery to the cell can also be confirmed through the imaging process with the use of appropriate cell labeling reagents. The ability to image the lipid-encapsulated particles delivering the agent provides for identification and/or confirmation of the cells or tissue to which the agent is delivered. Such particles would permit, for example, the site to be imaged in order to monitor the progress of the therapy on the site and to make desired adjustments in the dosage or therapeutic agent subsequently directed to the site, or to make adjustments to the frequency and/or amplitude of ultrasound pulsation to assure enhanced agent delivery to the target cell or tissue. In some instances, clinical transducers can be used to simultaneously image and enhance lipid exchange between the target and the lipid-encapsulated particle.

The invention thus provides a noninvasive means for the therapeutic treatment of thrombi, infarction, infection, cancers, atherosclerosis and inflammatory conditions, for example, in patients while employing conventional imaging equipment.

Methods of the invention are of use in delivery of therapeutic agents to, for example, cardiovascular-related tissues, including, but not limited to, heart tissue and all cardiovascular vessels, angiogenic tissue, any part of a cardiovascular vessel, any material or cell that comes into or caps cardiovascular a vessel, e.g., thrombi, clot or ruptured clot, platelets, muscle cells and the like. Disease conditions to be treated using the methods of the invention include, but are not limited to, any disease condition in which vasculature plays an important part in pathology, for example, cardiovascular disease, cancer, areas of inflammation, which may characterize a variety of disorders including rheumatoid arthritis, areas of irritation such as those affected by angioplasty resulting in restenosis, tumors, and areas affected by atherosclerosis. Depending upon the targeting ligand used, lipid-encapsulated particles together with ultrasound energy of the invention are of particular use in ameliorating symptoms associated with vascular and/or restenosis pathology. For example, lipid-encapsulated particles containing a ligand that binds to αᵥβ₃ integrin are targeted to tissues containing high expression levels of αᵥβ₃ integrin. High expression levels of αᵥβ₃ are typical of activated endothelial cells and are considered indicative of neovasculature. Directing ultrasound energy of appropriate frequency and amplitude to the particle located at the tissues containing high levels of αᵥβ₃ integrin results in enhanced delivery of the therapeutic agent from the particle to the targeted tissue as compared to agent delivery with the particle alone. Other tissues of interest to be treated include those containing particular malignant tissue and/or tumors, and tissues exhibiting inflammatory responses such as arthritis, vasculitis, or autoimmune diseases.

The lipid-encapsulated particles described herein are useful in the methods of the invention. The lipid-encapsulated particles may be targeted to a particular cell type and/or tissue through the use of ligands directed to the cell and/or tissue on the surface of the particles. The lipid-encapsulated particles and ultrasound energy can be used with cells or tissues in vivo, ex vivo, in situ and in vitro. For example, ultrasound energy applied to the targeted nongaseous, lipid-encapsulated particles can be used to deliver genetic material to cells, e.g., stem cells, and/or to label cells, e.g., stem cells, ex vivo or in vitro before implantation or further use of the cells.

Methods of administering the lipid-encapsulated particles of the invention in vivo and in vitro are well known to those in the art. The lipid-encapsulated particles of the present invention are administered, for example, by intravenous injection. In some instances, the particles are administered by infusion at a rate of approximately 3 μL/kg/min. In some embodiments, the lipid-encapsulated particles may be administered locally by, for example, catheter instillation at a particular site, and the ultrasound energy provided through transcutaneous isonification at the site of particle delivery. Although the particles are typically administered to target the vasculature, after administration, particles may go outside of the vasculature and reach additional cells and/or tissue. After administration of the lipid-encapsulated particles containing a therapeutic agent, known techniques for delivery of clinical levels of ultrasound energy are used to enhance delivery of the therapeutic agent to the targeted cells or tissue. If imaging is performed, known techniques of sonography can be used. Imaging also can be performed by MRI, nuclear, optical, CT, or PET methods if appropriate formulations are produced in concert with the therapeutic delivery.

Lipid-Encapsulated Particle Compositions

The lipid-encapsulated particle for use in the methods of the invention include nanoparticle emulsion as has been described, for example, in U.S. Pat. Nos. 5,780,010, 5,958,371 and 5,989,520). The nanoparticle emulsions are comprised of at least two immiscible liquids which are intimately dispersed, preferably, a hydrophobic material such as an oil, dispersed in water. The emulsions are in the form of droplets or nanoparticles having a diameter which typically is about 0.2 μm. Additives such as surface-active agents or finely-divided solids can be incorporated into the emulsion nanoparticles to increase their stability. The nanoparticles have a lipid monolayer bounding the hydrophobic core.

Fluorocarbon emulsions and, in particular, perfluorocarbon emulsions are well suited for biomedical applications and for use in the practice of the present invention. The perfluorocarbon emulsions are known to be stable, biologically inert and readily metabolized, primarily by trans-pulmonic alveolae evaporation. Further, their small particle size easily accommodates transpulmonic passage and their circulatory half-life (“beta elimination” half time: 1-2 hours) advantageously exceeds that of other agents. Furthermore, they are stable to ultrasound insonification indefinitely at all clinical power settings as compared with microbubbles which burst upon exposure to moderate to high ultrasound energy levels. Also, perfluorocarbons have been used to date in a wide variety of biomedical applications, including use as artificial blood substitutes. For use in the present invention, various fluorocarbon emulsions may be employed including those in which the fluorocarbon is a fluorocarbon-hydrocarbon, a perfluoroalkylated ether, polyether or crown ether. Useful perfluorocarbon emulsions are disclosed in U.S. Pat. Nos. 4,927,623, 5,077,036, 5,114,703, 5,171,755, 5,304,325, 5,350,571, 5,393,524, and 5,403,575 and include those in which the perfluorocarbon compound is perfluorotributylamine, perfluorodecalin, perfluorooctylbromide, perfluorodichlorooctane, perfluorooctane, perfluorodecane, perfluorotripropylamine, perfluorotrimethylcyclohexane or other perfluorocarbon compounds. Further, mixtures of such perfluorocarbon compounds may be incorporated in the emulsions utilized in the practice of the invention, as long as such mixtures do not result in phase conversion to gaseous perfluorcarbons for purposes of therapeutic delivery.

Emulsifying agents, for example surfactants, are used to facilitate the formation of emulsions and increase their stability. Typically, aqueous phase surfactants have been used to facilitate the formation of oil-in-water emulsions. A surfactant is any substance that contains both hydrophilic and a hydrophobic portions. When added to water or solvents, a surfactant reduces the surface tension.

The oil phase of the oil-in-water emulsion comprises, for example, 5 to 50% and, in some instances, 20 to 40% by weight of the emulsion. In some embodiments, the oil phase may comprise fatty acid esters such as triacylglycerol (corn oil). In some embodiments, the oil or hydrophobic constituent is a fluorochemical liquid. The fluorochemical liquid includes straight, branched chain and cyclic perfluorocarbons, straight, branched chain and cyclic perfluoro tertiary amines, straight, branched chain and cyclic perfluoro ethers and thioethers, chlorofluorocarbons and polymeric perfluoro ethers and the like. Although up to 50% hydrogen-substituted compounds can be used, perhalo compounds are preferred. Most preferred are perfluorinated compounds. Any fluorochemical liquid, i.e. a substance which is a liquid at or above body temperature (e.g. 37° C.) at atmospheric pressure, can be used to prepare a fluorochemical emulsion of the present invention. However, for many purposes emulsions fluorochemicals with longer extended stability are preferred. In order to obtain such emulsions, fluorochemical liquids with boiling points above 50° C. can be used, and in some cases, fluorochemical liquids with boiling points above about 80° C. can be used. The guiding determinant should be that the oil, e.g. a fluorochemical, should be expected to remain in a liquid phase (less than 0% gas conversion) under the intended conditions.

When the lipid encapsulated particles are constituted by a liposome rather than an emulsion, such a liposome may be prepared as generally described in the literature (see, for example, Kimelberg et al., CRC Crit. Rev. Toxicol. 6:25, 1978; Yatvin et al., Medical Physics 9:149, 1982). Liposomes are known to the art and generally comprise lipid materials including lecithin and sterols, egg phosphatidyl choline, egg phosphatidic acid, cholesterol and alpha-tocopherol.

Liposomes are small vesicles composed of an aqueous medium surrounded by lipids arranged in spherical bilayers. Liposomes are usually classified as small unilamellar vesicles (SUV), large unilamellar vesicles (LUV), or multi-lamellar vesicles (MLV). SUVs and LUVs, by definition, have only one lipid bilayer, whereas MLVs contain many concentric bilayers. Liposomes may be used to encapsulate various therapeutic agents and materials, by trapping hydrophilic molecules in the aqueous interior or between bilayers, or by trapping hydrophobic molecules within the bilayer.

The composition of the lipid bilayer, forming the structural basis for the liposome is generally composed at least of phospholipids, and more generally of mixtures of phospholipids with lipids per se. For example, in the liposomes, phosphatidylcholine derivatives, phosphatidylglycerol derivatives and the like are used along with non phospholipid components, if desired, such as cholesterol. Suitable alternative embodiments include mixtures of phospholipids with, for example, triglycerides. In addition, fatty acids, lipid vitamins, steroids, lipophilic drugs and other lipophilic compounds that can be included in a stable lipid bilayer which either do or do not include phospholipids can be used. Other lipids for use in liposomes include, for example, diacylglycerols. Liposomes of the invention may also contain therapeutic lipids, which include ether lipids, phosphatidic acid, phosphonates, ceramide and ceramide analogues, sphingosine and sphingosine analogues and serine-containing lipids. For suitable lipids see e.g., Lasic (1993) “Liposomes: from Physics to Applications” Elsevier, Amsterdam. Liposomes in general are referred to as smectic mesophases.

In some liposome embodiments, phospholipids are included and the liposomes may carry a net positive charge, a net negative charge or can be neutral. Inclusion of diacetylphosphate is a convenient method for conferring negative charge; stearylamine can be used to provide a positive charge. In some instances, at least one head group of the phospholipids is a phosphocholine, a phosphoethanolamine, a phosphoglycerol, a phosphoserine, or a phosphoinositol.

In some embodiments, the nongaseous, lipid-encapsulated particle is a lipid micelle or a lipoprotein micelle. Micelles are self-assembling particles composed of amphipathic lipids or polymeric components that are utilized for the delivery of sparingly soluble agents present in the hydrophobic core. Various means for the preparation of micellar delivery vehicles are available and may be carried out with ease by one skilled in the art. For instance, lipid micelles may be prepared as described in Perkins et al. (2000) Int. J. Pharm. 200:27-39. Lipoprotein micelles can be prepared from natural or artificial lipoproteins including low and high-density lipoproteins and chylomicrons.

In some embodiments, the nongaseous, lipid-encapsulated particle is a lipid encapsulated nanoparticle or microparticle which comprises a polymeric shell (nanocapsule), a polymer matrix (nanosphere) or a block copolymer, which may be cross-linked or else surrounded by a lipid layer or bilayer. Such lipid encapsulated nanoparticles and microparticles further comprise a therapeutic agent within the shell, dispersed throughout the matrix and/or within a hydrophobic core. General methods of preparing such nanoparticles and microparticles are described in the art, for example, in Soppimath et al. (2001) J. Control Release 70:1-20 and Allen et al. (2000) J. Control Release 63:275-286. For example, polymers such as polycaprolactone and poly(d,l-lactide) may be used while the lipid layer is composed of a mixture of lipid as described herein. Derivatized single chain polymers are polymers adapted for covalent linkage of a biologically active agent to form a polymer-agent conjugate. Numerous polymers have been proposed for synthesis of polymer-agent conjugates including polyaminoacids, polysaccharides such as dextrin or dextran, and synthetic polymers such as N-(2-hydroxypropyl)methacrylamide (HPMA) copolymer. Suitable methods of preparation are described in the art, for example, in Veronese et al. (1999) IL Farmaco 54:497-516. Other suitable polymers can be any known in the art of pharmaceuticals and include, but are not limited to, naturally-occurring polymers such as hydroxyethyl starch, proteins, glycopeptides and lipids. The synthetic polymers can also be linear or branched, substituted or unsubstituted, homopolymeric, co-polymers, or block co-polymers of two or more different synthetic monomers.

In a specific example, the lipid encapsulated particles may be constituted by a perfluorocarbon emulsion, the particles having an outer coating of a derivatized natural or synthetic phospholipid, a fatty acid, cholesterol, lipid, sphingomyelin, tocopherol, glucolipid, sterylamine, cardiolipin, a lipid with ether or ester linked fatty acids or a polymerized lipid.

As a specific example of a perfluorocarbon emulsion useful in the invention may be mentioned a perfluorodichlorooctane or perfluorooctylbromide emulsion wherein the lipid coating thereof contains between approximately 50 to 99.5 mole percent lecithin, preferably approximately 55 to 70 to mole percent lecithin, 0 to 50 mole percent cholesterol, preferably approximately 25 to 45 mole percent cholesterol and approximately 0.5 to 10 mole percent biotinylated phosphatidylethanolamine, preferably approximately 1 to 5 mole percent biotinylated phosphatidylethanolamine. Other phospholipids such as phosphatidylserine may be biotinylated, fatty acyl groups such as stearylamine may be conjugated to biotin, or cholesterol or other fat soluble chemicals may be biotinylated and incorporated in the lipid coating for the lipid encapsulated particles. The preparation of an exemplary biotinylated perfluorocarbon for use in the practice of the invention is described in accordance with known procedures.

Reference to the term “nongaseous” or “liquid” in the context of the lipid-encapsulated particles of the present invention is generally intended to mean that the interior volume of the particles contains no gas phase. In some instances, less than about 2% of the interior volume of the particles is in a gas phase per total volume of the particles (i.e. v/v), in some instances, no more than about 1% (v/v). The term “about” as used herein is intended to encompass a range of values 10% above and below a stated value such that, for example, about 1% is intended to encompass the range of values from 0.9% to 1.1%. The non-gaseous nature of the particles is such that when ultrasound is applied to effect drug delivery, insufficient gas is present in the particles to disrupt the lipid encapsulating layer. Thus, “non-gaseous” is defined accordingly.

It is understood that the “lipid membrane” or “lipid bilayer” or “lipid monolayer” of lipid-encapsulated particles need not consist exclusively of lipids, but can additionally contain any suitable other components, including, but not limited to, cholesterol and other steroids, lipid-soluble chemicals, proteins of any length, and other amphipathic molecules. For those particles with a lipid monolayer, the general structure of the membrane is a single hydrophilic surface bounding a hydrophobic core. For those particles with a lipid bilayer, the general structure of the membrane is a sheet of two hydrophilic surfaces sandwiching a hydrophobic core. For a general discussion of membrane structure, see The Encyclopedia of Molecular Biology by J. Kendrew (1994).

The term “ligand” as used herein is intended to refer to a targeting molecule that binds specifically to another molecule of a biological target separate and distinct from the particle itself. The reaction does not require nor exclude a molecule that donates or accepts a pair of electrons to form a coordinate covalent bond with a metal atom of a coordination complex. Thus a ligand may be attached covalently for direct-conjugation or noncovalently for indirect conjugation to the surface of the particle surface.

Useful lipid-encapsulated particles, for example, may have a wide range of nominal particle diameters, e.g., from as small as about 0.01 μm to as large as 10 μm, preferably about 50 nm to about 1000 nm, more preferably about 50 nm to about 500 nm, in some instances about 50 nm to about 300 nm, in some instances about 100 nm to about 300 nm, in some instances about 200 nm to about 250 nm, in some instances about 200 nm, in some instances about less than 200 nm. Generally, small size particles, for example, submicron particles, circulate longer and tend to be more stable than larger particles.

The lipid/surfactants used to form an outer coating on the particles (that can contain the coupled ligand or entrap reagents for binding desired components to the surface) include natural or synthetic phospholipids, fatty acids, cholesterols, lysolipids, sphingomyelins, tocopherols, glucolipids, stearylamines, cardiolipins, plasmalogens, a lipid with ether or ester linked fatty acids, and polymerized lipids. In some instances, the lipid/surfactant can include lipid conjugated polyethylene glycol (PEG). Various commercial anionic, cationic, and nonionic surfactants can also be employed, including Tweens, Spans, Tritons, and the like. In some embodiments, preferred surfactants are phospholipids and cholesterol.

Fluorinated surfactants which are soluble in the oil to be emulsified can also be used. Suitable fluorochemical surfactants include perfluorinated alkanoic acids such as perfluorohexanoic and perfluorooctanoic acids and amidoamine derivatives. These surfactants are generally used in amounts of 0.01 to 5.0% by weight, and preferably in amounts of 0.1 to 1.0%. Other suitable fluorochemical surfactants include perfluorinated alcohol phosphate esters and their salts; perfluorinated sulfonamide alcohol phosphate esters and their salts; perfluorinated alkyl sulfonamide; alkylene quaternary ammonium salts; N,N(carboxyl-substituted lower alkyl) perfluorinated alkyl sulfonamides; and mixtures thereof. As used herein, the term “perfluorinated” means that the surfactant contains at least one perfluorinated alkyl group.

Suitable perfluorinated alcohol phosphate esters include the free acids of the diethanolamine salts of mono- and bis(1H, 1H, 2H, 2H-perfluoroalkyl)phosphates. The phosphate salts, available under the tradename ZONYL RP (Dupont, Wilmington, Del.), are converted to the corresponding free acids by known methods. Suitable perfluorinated sulfonamide alcohol phosphate esters are described in U.S. Pat. No. 3,094,547. Suitable perfluorinated sulfonamide alcohol phosphate esters and salts of these include perfluoro-n-octyl-N-ethylsulfonamidoethyl phosphate, bis(perfluoro-n-octyl-N-ethylsulfonamidoethyl)phosphate, the ammonium salt of bis(perfluoro-n-octyl-N-ethylsulfonamidoethyl) phosphate,bis(perfluorodecyl-N-ethylsulfonamidoethyl)-phosphate and bis(perfluorohexyl-N ethylsulfonamidoethyl)phosphate. The preferred formulations use phosphatidylcholine, derivatized-phosphatidylethanolamine and cholesterol as the lipid surfactant.

Other known surfactant additives such as PLURONIC F-68, HAMPOSYL L30 (W.R. Grace Co., Nashua, N.H.), sodium dodecyl sulfate, Aerosol 413 (American Cyanamid Co., Wayne, N.J.), Aerosol 200 (American Cyanamid Co.), LIPOPROTEOL LCO (Rhodia Inc., Mammoth, N.J.), STANDAPOL SH 135 (Henkel Corp., Teaneck, N.J.), FIZUL 10-127 (Finetex Inc., Elmwood Park, N.J.), and CYCLOPOL SBFA 30 (Cyclo Chemicals Corp., Miami, Fla.); amphoterics, such as those sold with the trade names: DERIPHAT 170 (Henkel Corp.), LONZAINE JS (Lonza, Inc.), NIRNOL C2N-SF (Miranol Chemical Co., Inc., Dayton, N.J.), AMPHOTERGE W2 (Lonza, Inc.), and AMPHOTERGE 2WAS (Lonza, Inc.); non-ionics, such as those sold with the trade names: PLURONIC F-68 (BASF Wyandotte, Wyandotte, Mich.), PLURONIC F-127 (BASF Wyandotte), BRIJ 35 (ICI Americas; Wilmington, Del.), TRITON X-100 (Rohm and Haas Co., Philadelphia, Pa.), BRIJ 52 (ICI Americas), SPAN 20 (ICI Americas), GENEROL 122 ES (Henkel Corp.), TRITON N-42 (Rohm and Haas Co.), TRITON N-101 (Rohm and Haas Co.), TRITON X-405 (Rohm and Haas Co.), TWEEN 80 (ICI Americas), TWEEN 85 (ICI Americas), and BRIJ 56 (ICI Americas) and the like, may be used alone or in combination in amounts of 0.10 to 5.0% by weight to assist in stabilizing the emulsions.

Lipid encapsulated particles may be formulated with cationic lipids in the surfactant layer that facilitate entrapping or adhering ligands, such as nucleic acids and aptamers, to particle surfaces. Typical cationic lipids may include DOTMA, N-[1-(2,3-dioleoyloxy)propyl]-N,N,N-trimethylammonium chloride; DOTAP, 1,2-dioleoyloxy-3-(trimethylammonio)propane; DOTB, 1,2-dioleoyl-3-(4′-trimethyl-ammonio)butanoyl-sn-glycerol, 1,2-diacyl-3-trimethylammonium-propane; DAP, 1,2-diacyl-3-dimethylammonium-propane; TAP, 1,2-diacyl-3-trimethylammonium-propane; 1,2-diacyl-sn-glycerol-3-ethyl phosphocholine; 3β-[N′,N′-dimethylaminoethane)-carbamol]cholesterol-HCl, DC-Cholesterol (DC-Chol); and DDAB, dimethyldioctadecylammonium bromide. In general the molar ratio of cationic lipid to non-cationic lipid in the lipid surfactant monolayer may be, for example, 1:1000 to 2:1, preferably, between 2:1 to 1:10, more preferably in the range between 1:1 to 1:2.5 and most preferably 1:1 (ratio of mole amount cationic lipid to mole amount non-cationic lipid, e.g., DPPC). A wide variety of lipids may comprise the non-cationic lipid component of the surfactant, particularly dipalmitoylphosphatidylcholine, dipalmitoylphosphatidyl-ethanolamine or dioleoylphosphatidylethanolamine in addition to those previously described. In lieu of cationic lipids as described above, lipids bearing cationic polymers such as polylysine or polyarginine may also be included in the lipid surfactant and afford binding of a negatively charged therapeutic, such as genetic material or analogues there of, to the outside of the emulsion particles. Although the lipids can be cross-linked to provide stability to the particles for use in vivo, doing so may be disadvantageous since cross-linking may inhibit the lipid components from freely flowing out into the cells with which they fuse. Accordingly, it is preferable that the lipid components of the particles are not cross-linked.

In particular embodiments, included in the lipid/surfactant coating are components with reactive groups that can be used to couple a targeting ligand and/or the ancillary substance useful for therapy. In some embodiments, a lipid/surfactant coating which provides a vehicle for binding a multiplicity of copies of one or more desired components to the particle is preferred. As will be described below, the lipid/surfactant components can be coupled to these reactive groups through functionalities contained in the lipid/surfactant component. For example, phosphatidylethanolamine may be coupled through its amino group directly to a desired moiety, or may be coupled to a linker such as a short peptide which may provide carboxyl, amino, or sulfhydryl groups as described below. Alternatively, standard linking agents such a maleimides may be used. A variety of methods may be used to associate the targeting ligand and the ancillary substances to the particles; these strategies may include the use of spacer groups such as polyethyleneglycol or peptides, for example.

For example, lipid/surfactant coated nanoparticles are typically formed by microfluidizing a mixture of the oil which forms the core and the lipid/surfactant mixture which forms the outer layer in suspension in aqueous medium to form an emulsion. In this procedure, the lipid/surfactants may already be coupled to additional ligands when they are emulsified into the nanoparticles, or may simply contain reactive groups for subsequent coupling. Alternatively, the components to be included in the lipid/surfactant layer may simply be solubilized in the layer by virtue of the solubility characteristics of the ancillary material. Sonication or other techniques may be required to obtain a suspension of the lipid/surfactant in the aqueous medium. Typically, at least one of the materials in the lipid/surfactant outer layer comprises a linker or functional group which is useful to bind the additional desired component or the component may already be coupled to the material at the time the emulsion is prepared.

The covalent linking of the targeting ligands to the materials in the lipid-encapsulated particles may be accomplished using synthetic organic techniques which would be readily apparent to one of ordinary skill in the art based on the present disclosure. For example, the targeting ligand may be linked to the material, including the lipid, via the use of well known coupling or activation agents.

For coupling by covalently binding the targeting ligand or other organic moiety to the components of the outer layer, various types of bonds and linking agents may be employed. Typical methods for forming such coupling include formation of amides with the use of carbodiamides, or formation of sulfide linkages through the use of unsaturated components such as maleimide. Other coupling agents include, for example, glutaraldehyde, propanedial or butanedial, 2-iminothiolane hydrochloride, bifunctional N-hydroxysuccinimide esters such as disuccinimidyl suberate, disuccinimidyl tartrate, bis[2-(succinimidooxycarbonyloxy)ethyl]sulfone, heterobifunctional reagents such as N-(5-azido-2-nitrobenzoyloxy)succinimide, succinimidyl 4-(N-maleimidomethyl)cyclohexane-1-carboxylate, and succinimidyl 4-(p-maleimidophenyl)butyrate, homobifunctional reagents such as 1,5-difluoro-2,4-dinitrobenzene, 4,4′-difluoro-3,3′-dinitrodiphenylsulfone, 4,4′-diisothiocyano-2,2′-disulfonic acid stilbene, p-phenylenediisothiocyanate, carbonylbis(L-methionine p-nitrophenyl ester), 4,4′-dithiobisphenylazide, erythritolbiscarbonate and bifunctional imidoesters such as dimethyl adipimidate hydrochloride, dimethyl suberimidate, dimethyl 3,3′-dithiobispropionimidate hydrochloride and the like. Linkage can also be accomplished by acylation, sulfonation, reductive amination, and the like. A multiplicity of ways to couple, covalently, a desired ligand to one or more components of the outer layer is well known in the art. The ligand itself may be included in the surfactant layer if its properties are suitable. For example, if the ligand contains a highly lipophilic portion, it may itself be embedded in the lipid/surfactant coating. Further, if the ligand is capable of direct adsorption to the coating, this too will effect its coupling. For example, nucleic acids, because of their negative charge, adsorb directly to cationic surfactants.

The covalent bonds may involve crosslinking and/or polymerization. Crosslinking generally refers to the attachment of two chains of polymer molecules by bridges, composed of either an element, a group, or a compound, which join certain carbon atoms of the chains by covalent chemical bonds. For example, crosslinking may occur in polypeptides which are joined by the disulfide bonds of the cystine residue. Crosslinking may be achieved, for example, by (1) adding a chemical substance (cross-linking agent) and exposing the mixture to heat, or (2) subjecting a polymer to high energy radiation.

Noncovalent associations can also occur through ionic interactions involving a targeting ligand and residues within a moiety on the surface of the lipid-encapsulated particle. Noncovalent associations can also occur through ionic interactions involving a targeting ligand and residues within a primer, such as charged amino acids, or through the use of a primer portion comprising charged residues that can interact with both the targeting ligand and the lipid-encapsulated particle surface. For example, noncovalent conjugation can occur between a generally negatively-charged targeting ligand or moiety on a lipid-encapsulated particle surface and positively-charged amino acid residues of a primer, e.g., polylysine, polyarginine and polyhistidine residues.

The ligand may bind directly to the particle, i.e., the ligand is associated with the particle itself. Alternatively, indirect binding may also be effected using a hydrolizable anchor, such as a hydrolizable lipid anchor, to couple the targeting ligand or other organic moiety to the lipid/surfactant coating of the particle. Indirect binding such as that effected through biotin/avidin may also be employed for the ligand. For example, in biotin/avidin mediated targeting, the targeting ligand is coupled not to the particle, but rather coupled, in biotinylated form to the targeted tissue.

Ancillary agents that may be coupled to the lipid-encapsulated particles through entrapment in the coating layer include radionuclides. Radionuclides may be either therapeutic or diagnostic; diagnostic imaging using such nuclides is well known and by targeting radionuclides to desired tissue a therapeutic benefit may be realized as well. Radionuclides for diagnostic imaging often include gamma emitters (e.g., ⁹⁶Tc) and radionuclides for therapeutic purposes often include alpha emitters (e.g., ²²⁵Ac) and beta emitters (e.g., ⁹⁰Y). Typical diagnostic radionuclides include ^{99m}Tc, ⁹⁶Tc, ⁹⁵Tc, ¹¹¹In, ⁶²Cu, ⁶⁴Cu, ⁶⁷Ga, ⁶⁸Ga, and ¹⁹²Ir, and therapeutic nuclides include ²²⁵Ac, ¹⁸⁶Re, ¹⁸⁸Re, ¹⁵³Sm, ¹⁶⁶Ho, ¹⁷⁷Lu, ¹⁴⁹Pm, ⁹⁰Y, ²¹²Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ¹²³I, ¹³¹I, ⁶⁷Cu, ¹⁰⁵Rh, ¹¹¹Ag, and ¹⁹²Ir. The nuclide can be provided to a preformed particle in a variety of ways. For example, ⁹⁹Tc-pertechnate may be mixed with an excess of stannous chloride and incorporated into the preformed emulsion of nanoparticles. Stannous oxinate can be substituted for stannous chloride. In addition, commercially available kits, such as the HM-PAO (exametazine) kit marketed as Ceretek® by Nycomed Amersham can be used. Means to attach various radioligands to the lipid-encapsulated particles of the invention are understood in the art.

Chelating agents containing metal ions for use, for example, in magnetic resonance imaging can also be employed as ancillary agents. Typically, a chelating agent containing a paramagnetic metal or superparamagnetic metal is associated with the lipids/surfactants of the coating on the particles and incorporated into the initial mixture. The chelating agent can be coupled directly to one or more of components of the coating layer. Suitable chelating agents are macrocyclic or linear chelating agents and include a variety of multi-dentate compounds including EDTA, DPTA, DOTA, and the like. These chelating agents can be coupled directly to functional groups contained in, for example, phosphatidyl ethanolamine, oleates, or any other synthetic natural or functionalized lipid or lipid soluble compound. Alternatively, these chelating agents can coupled through linking groups.

Chelating agents appropriate for use in some instances include 1,4,7,10-tetraazacyclododecane-1,4,7,10-tetraacetic acid (DOTA) and its derivatives, in particular, a methoxybenzyl derivative (MEO-DOTA) and a methoxybenzyl derivative comprising an isothiocyanate functional group (MEO-DOTA-NCS) which can then be coupled to the amino group of phosphatidyl ethanolamine or to a peptide derivatized form thereof. Derivatives of this type are described in U.S. Pat. No. 5,573,752 and other suitable chelating agents are disclosed in U.S. Pat. No. 6,056,939.

The DOTA isocyanate derivative can also be coupled to the lipid/surfactant directly or through a peptide spacer. The use of gly-gly-gly as a spacer is illustrated in the reaction scheme below. For direct coupling, the MEO-DOTA-NCS is simply reacted with phosphoethanolamine (PE) to obtain the coupled product. When a peptide is employed, for example a triglycyl link, PE is first coupled to t-boc protected triglycine. Standard coupling techniques, such as forming the activated ester of the free acid of the t-boc-triglycine using diisopropyl carbodiimide (or an equivalent thereof) with either N-hydroxy succinimide (NHS) or hydroxybenzotriazole (HBT) are employed and the t-boc-triglycine-PE is purified.

Treatment of the t-boc-triglycine-PE with trifluoroacetic acid yields triglycine-PE, which is then reacted with excess MEO-DOTA-NCS in DMF/CHCl₃ at 50° C. The final product is isolated by removing the solvent, followed by rinsing the remaining solid with excess water, to remove excess solvent and any un-reacted or hydrolyzed MEO-DOTA-NCS.

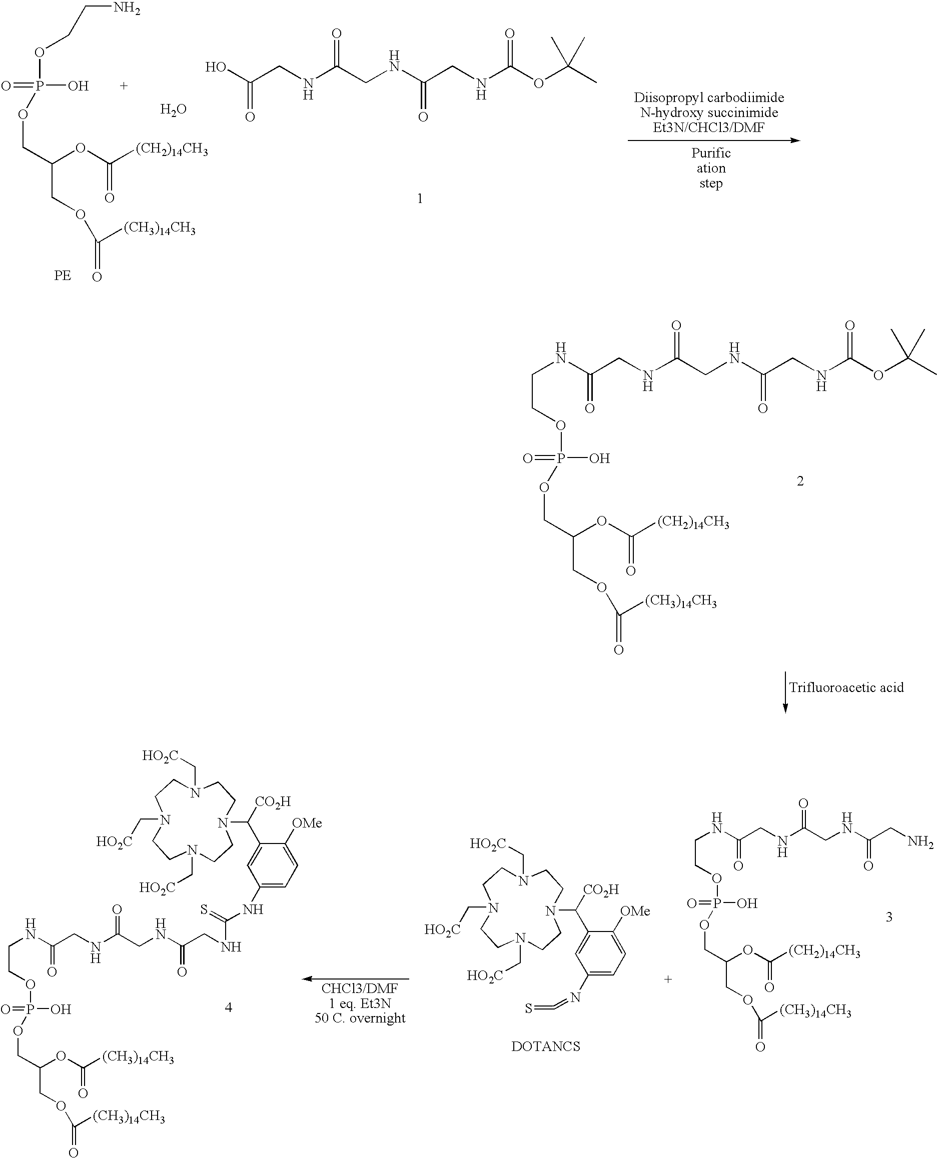

Other ancillary agents include fluorophores (such as fluorescein, dansyl, quantum dots, and the like) and infrared dyes or metals may be used in optical or light imaging (e.g., confocal microscopy and fluorescence imaging). For nuclear imaging, such as PET imaging, tosylated and ¹⁸F fluorinated compounds may be associated with the nanoparticles as ancillary agents.

In all of the foregoing cases, whether the associated moiety is a targeting ligand or is an ancillary agent, the defined moiety may be non-covalently associated with the lipid/surfactant layer, may be directly coupled to the components of the lipid/surfactant layer, or may be indirectly coupled to said components through spacer moieties.

The therapeutic target may be an in vivo or in vitro target and, preferably, a biological material although the target need not be a biological material. The target may be comprised of a surface to which the contrast substance binds or a three dimensional structure in which the contrast substance penetrates and binds to portions of the target below the surface.

The targeting ligand coupled to the surface of the particles is generally specific for a desired target to allow active targeting. Active targeting refers to ligand-directed, site-specific accumulation of agents to cells, tissues or organs by localization and binding to molecular epitopes, e.g., receptors, lipids, peptides, cell adhesion molecules, polysaccharides, biopolymers, and the like, presented on the surface membranes of cells or within the extracellular or intracellular matrix. A wide variety of ligands can be used including an antibody, a fragment of an antibody, a polypeptide such as small oligopeptide, a large polypeptide or a protein having three dimensional structure, a peptidomimetic, a polysaccharide, an aptamer, a lipid, a nucleic acid, a lectin or a combination thereof. Generally, the ligand specifically binds to a cellular epitope or receptor.

In some embodiments, for example for use in vivo, the binding affinity of the ligand for its specific target is about 10⁻⁷ M or greater. In some embodiments, for example, for use in vitro, the binding affinity of the ligand for its specific target can be less than 10⁻⁷ M.

Avidin-biotin interactions are extremely useful, noncovalent targeting systems that have been incorporated into many biological and analytical systems and selected in vivo applications. Avidin has a high affinity for biotin (10⁻¹⁵ M) facilitating rapid and stable binding under physiological conditions. Some targeted systems utilizing this approach are administered in two or three steps, depending on the formulation. Typically in these systems, a biotinylated ligand, such as a monoclonal antibody, is administered first and “pretargeted” to the unique molecular epitopes. Next, avidin is administered, which binds to the biotin moiety of the “pretargeted” ligand. Finally, the biotinylated emulsion is added and binds to the unoccupied biotin-binding sites remaining on the avidin thereby completing the ligand-avidin-emulsion “sandwich.” The avidin-biotin approach can avoid accelerated, premature clearance of targeted agents by the reticuloendothelial system secondary to the presence of surface antibody. Additionally, avidin, with four, independent biotin binding sites provides signal amplification and improves detection sensitivity.

As used herein, the term “biotin emulsion” or “biotinylated” with respect to conjugation to a biotin emulsion or biotin agent is intended to include biotin, biocytin and other biotin derivatives and analogs such as biotin amido caproate N-hydroxysuccinimide ester, biotin 4-amidobenzoic acid, biotinamide caproyl hydrazide and other biotin derivatives and conjugates. Other derivatives include biotin-dextran, biotin-disulfide N-hydroxysuccinimide ester, biotin-6 amido quinoline, biotin hydrazide, d-biotin-N hydroxysuccinimide ester, biotin maleimide, d-biotin p-nitrophenyl ester, biotinylated nucleotides and biotinylated amino acids such as N, epsilon-biotinyl-1-lysine. The term “avidin emulsion” or “avidinized” with respect to conjugation to an avidin emulsion or avidin agent is intended to include avidin, streptavidin and other avidin analogs such as streptavidin or avidin conjugates, highly purified and fractionated species of avidin or streptavidin, and non-amino acid or partial-amino acid variants, recombinant or chemically synthesized avidin.

Targeting ligands may be chemically attached to the surface of lipid-encapsulated particles by a variety of methods depending upon the nature of the particle surface. Conjugations may be performed before or after the emulsion particle is created depending upon the ligand employed. Direct chemical conjugation of ligands to proteinaceous agents often take advantage of numerous amino-groups (e.g. lysine) inherently present within the surface. Alternatively, functionally active chemical groups such as pyridyldithiopropionate, maleimide or aldehyde may be incorporated into the surface as chemical “hooks” for ligand conjugation after the particles are formed. Another common post-processing approach is to activate surface carboxylates with carbodiimide prior to ligand addition. The selected covalent linking strategy is primarily determined by the chemical nature of the ligand. Antibodies and other large proteins may denature under harsh processing conditions; whereas, the bioactivity of carbohydrates, short peptides, aptamers, drugs or peptidomimetics often can be preserved. To ensure high ligand binding integrity and maximize targeted particle avidity flexible polymer spacer arms, e.g. polyethylene glycol or simple caproate bridges, can be inserted between an activated surface functional group and the targeting ligand. These extensions can be 10 nm or longer and minimize interference of ligand binding by particle surface interactions.

Antibodies, particularly monoclonal antibodies, may also be used as site-targeting ligands directed to any of a wide spectrum of molecular epitopes including pathologic molecular epitopes. Immunoglobin-γ (IgG) class monoclonal antibodies have been conjugated to liposomes, emulsions and other lipid-encapsulated particles to provide active, site-specific targeting. Generally, these proteins are symmetric glycoproteins (MW ca. 150,000 Daltons) composed of identical pairs of heavy and light chains. Hypervariable regions at the end of each of two arms provide identical antigen-binding domains. A variably sized branched carbohydrate domain is attached to complement-activating regions, and the hinge area contains particularly accessible interchain disulfide bonds that may be reduced to produce smaller fragments.

In some instances, monoclonal antibodies are used in the antibody compositions of the invention. Monoclonal antibodies specific for selected antigens on the surface of cells may be readily generated using conventional techniques (see, for example, U.S. Pat. Nos. RE 32,011, 4,902,614, 4,543,439, and 4,411,993). Hybridoma cells can be screened immunochemically for production of antibodies specifically reactive with an antigen, and monoclonal antibodies can be isolated. Other techniques may also be utilized to construct monoclonal antibodies (see, for example, Huse et al. (1989) Science 246:1275-1281; Sastry et al. (1989) Proc. Natl. Acad. Sci. USA 86:5728-5732; Alting-Mees et al. (1990) Strategies in Molecular Biology 3:1-9).

Within the context of the present invention, antibodies are understood to include various kinds of antibodies, including, but not necessarily limited to, naturally occurring antibodies, monoclonal antibodies, polyclonal antibodies, antibody fragments that retain antigen binding specificity (e.g., Fab, and F(ab′)₂) and recombinantly produced binding partners, single domain antibodies, hybrid antibodies, chimeric antibodies, single-chain antibodies, human antibodies, humanized antibodies, and the like. Generally, antibodies are understood to be reactive against a selected antigen of a cell if they bind with an affinity (association constant) of greater than or equal to 10⁷ M⁻¹. Antibodies against selected antigens for use with the emulsions may be obtained from commercial sources.

Further description of the various kinds of antibodies of use as site-targeting ligands in the invention is provided herein, in particular, later in this Lipid-Encapsulated Particle Compositions section.

The lipid-encapsulated particles of use in the present invention also employ targeting agents that are ligands other than an antibody or fragment thereof. For example, polypeptides, like antibodies, may have high specificity and epitope affinity for use as vector molecules for targeted contrast agents. These may be small oligopeptides, having, for example, 5 to 10 amino acid, specific for a unique receptor sequences (such as, for example, the RGD epitope of the platelet GIIbIIIa receptor) or larger, biologically active hormones such as cholecystokinin. Smaller peptides potentially have less inherent immunogenicity than nonhumanized murine antibodies. Peptides or peptide (nonpeptide) analogues of cell adhesion molecules, cytokines, selectins, cadhedrins, Ig superfamily, integrins and the like may be utilized for targeted therapeutic delivery.

In some instances, the ligand is a non-peptide organic molecule, such as those described in U.S. Pat. No. 6,130,231 (for example as set forth in formula 1); U.S. Pat. Nos. 6,153,628; 6,322,770; and PCT publication WO 01/97848. “Non-peptide” moieties in general are those other than compounds which are simply polymers of amino acids, either gene encoded or non-gene encoded. Thus, “non-peptide ligands” are moieties which are commonly referred to as “small molecules” lacking in polymeric character and characterized by the requirement for a core structure other than a polymer of amino acids. The non-peptide ligands useful in the invention may be coupled to peptides or may include peptides coupled to portions of the ligand which are responsible for affinity to the target site, but it is the non-peptide regions of this ligand which account for its binding ability. For example, non-peptide ligands specific for the αᵥβ₃ integrin are described in U.S. Pat. Nos. 6,130,231 and 6,153,628.

Carbohydrate-bearing lipids may be used for targeting of the lipid-encapsulated particles, as described, for example, in U.S. Pat. No. 4,310,505.

Asialoglycoproteins have been used for liver-specific applications due to their high affinity for asialoglycoproteins receptors located uniquely on hepatocytes. Asialoglycoproteins directed agents (primarily magnetic resonance agents conjugated to iron oxides) have been used to detect primary and secondary hepatic tumors as well as benign, diffuse liver disease such as hepatitis. The asialoglycoproteins receptor is highly abundant on hepatocytes, approximately 500,000 per cell, rapidly internalizes and is subsequently recycled to the cell surface. Polysaccharides such as arabinogalactan may also be utilized to localize emulsions to hepatic targets. Arabinogalactan has multiple terminal arabinose groups that display high affinity for asialoglycoproteins hepatic receptors.

Aptamers are high affinity, high specificity RNA or DNA-based ligands produced by in vitro selection experiments (SELEX: systematic evolution of ligands by exponential enrichment). Aptamers are generated from random sequences of 20 to 30 nucleotides, selectively screened by absorption to molecular antigens or cells, and enriched to purify specific high affinity binding ligands. To enhance in vivo stability and utility, aptamers are generally chemically modified to impair nuclease digestion and to facilitate conjugation with drugs, labels or particles. Other, simpler chemical bridges often substitute nucleic acids not specifically involved in the ligand interaction. In solution aptamers are unstructured but can fold and enwrap target epitopes providing specific recognition. The unique folding of the nucleic acids around the epitope affords discriminatory intermolecular contacts through hydrogen bonding, electrostatic interaction, stacking, and shape complementarity. In comparison with protein-based ligands, generally aptamers are stable, are more conducive to heat sterilization, and have lower immunogenicity. Aptamers are currently used to target a number of clinically relevant pathologies including angiogenesis, activated platelets, and solid tumors and their use is increasing. The clinical effectiveness of aptamers as targeting ligands for imaging and/or therapeutic emulsion particles may be dependent upon the impact of the negative surface charge imparted by nucleic acid phosphate groups on clearance rates. Previous research with lipid-based particles suggest that negative zeta potentials markedly decrease liposome circulatory half-life, whereas, neutral or cationic particles have similar, longer systemic persistence.

It is also possible to use what has been referred to as a “primer material” to couple specific binding species to the lipid-encapsulated particles for certain applications. As used herein, “primer material” refers to any constituent or derivatized constituent incorporated into the emulsion lipid surfactant layer that could be chemically utilized to form a covalent bond between the particle and a targeting ligand or a component of the targeting ligand such as a subunit thereof.

Thus, the targeting ligand may be immobilized on the encapsulating lipid monolayer by direct adsorption to the oil/aqueous interface or using a primer material. A primer material may be any surfactant compatible compound incorporated in the particle to chemically couple with or adsorb a specific binding or targeting species. For example, an emulsion can be formed with an aqueous continuous phase and a biologically active ligand adsorbed or conjugated to the primer material at the interface of the continuous and discontinuous phases. Naturally occurring or synthetic polymers with amine, carboxyl, mercapto, or other functional groups capable of specific reaction with coupling agents and highly charged polymers may be utilized in the coupling process. The specific binding species (e.g. antibody) may be immobilized on the emulsion particle surface by direct adsorption or by chemical coupling. Examples of specific binding species which can be immobilized by direct adsorption include small peptides, peptidomimetics, or polysaccharide-based agents. To make such an emulsion the specific binding species may be suspended or dissolved in the aqueous phase prior to formation of the emulsion. Alternatively, the specific binding species may be added after formation of the emulsion and incubated with gentle agitation at room temperature (about 25° C.) in a pH 7.0 buffer (typically phosphate buffered saline) for 1.2 to 18 hours.

Where the specific binding species is to be coupled to a primer material, conventional coupling techniques may be used. The specific binding species may be covalently bonded to primer material with coupling agents using methods which are known in the art. Primer materials may include phosphatidylethanolamine (PE), N-caproylamine-PE, n-dodecanylamine, phosphatidylthioethanol,N-1,2-diacyl-sn-glycero-3-phosphoethanolamine-N-[4-(p-maleimidophenyl)butyramide], 1,2-diacyl-sn-glycero-3-phosphoethanolamine-N-[4-(p-maleimidomethyl)cyclohexane-carboxylate], 1,2-diacyl-sn-glycero-3-phosphoethanolamine-N-[3-(2-pyridyldithio)propionate], 1,2-diacyl-sn-glycero-3-phosphoethanolamine-N[PDP(polyethylene glycol)2000], N-succinyl-PE, N-glutaryl-PE, N-dodecanyl-PE, N-biotinyl-PE, or N-caproyl-PE. Additional coupling agents include, for example, use a carbodiimide or an aldehyde having either ethylenic unsaturation or having a plurality of aldehyde groups. Further description of additional coupling agents appropriate for use is provided herein, in particular, later in this Lipid-Encapsulated Particle Compositions section.

Covalent bonding of a specific binding species to the primer material can be carried out with the reagents provided herein and with others by conventional, well-known reactions, for example, in the aqueous solutions at a neutral pH, at temperatures of less than 25° C. for 1 hour to overnight. Examples of linkers for coupling a ligand, including non-peptide ligands, are known in the art.

In certain embodiments, the targeting ligands may be incorporated in the present compositions via non-covalent associations. As known in the art, non-covalent association is generally a function of a variety of factors, including, for example, the polarity of the involved molecules, the charge (positive or negative), if any, of the involved molecules, the extent of hydrogen bonding through the molecular network, and the like. Non-covalent bonds are generally selected from the group consisting of ionic interaction, dipole-dipole interaction, hydrogen bonds, hydrophilic interactions, van der Waal's forces, and any combinations thereof.

Non-covalent interactions may be used to couple the target cell directed moiety to the lipid or directly to another component at the surface of the lipid-encapsulated particle. For example, the amino acid sequence Gly-Gly-His may be bound to the surface of an lipid-encapsulated particles, preferably by a primer material, such as PEG, and copper, iron or vanadyl ion may then be added. Proteins, such as antibodies which contain histidine residues, may then bind to the lipid-encapsulated particles via an ionic bridge with the copper ion, as described in U.S. Pat. No. 5,466,467. An example of hydrogen bonding involves cardiolipin lipids which can be incorporated into the lipid compositions. Examples of non-covalent associations can also occur through ionic interactions involving a targeting ligand and residues within a primer or on an lipid-encapsulated particle, such as charged amino acids, include those between a generally negatively-charged target cell directed moiety or moiety on an lipid-encapsulated particle surface and positively-charged amino acid residues of a primer, e.g., polylysine, polyarginine and polyhistidine residues.

The free end of the hydrophilic primer, such as polyethylene glycol ethylamine, which contains a reactive group, such as an amine or hydroxyl group, could be used to couple a target cell directed moiety. For example, polyethylene glycol ethylamine may be reacted with N-succinimidylbiotin or p-nitrophenylbiotin to introduce onto the spacer a useful coupling group. For example, biotin may be coupled to the spacer and this will readily bind non-covalently proteins or other target cell directed moieties bearing avidin or streptavidin.

Emulsifying and/or solubilizing agents may also be used in conjunction with emulsions. Such agents include, but are not limited to, acacia, cholesterol, diethanolamine, glyceryl monostearate, lanolin alcohols, lecithin, mono- and di-glycerides, mono-ethanolamine, oleic acid, oleyl alcohol, poloxamer, peanut oil, palmitic acid, polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, trolamine, and emulsifying wax. All lipids with perfluoro fatty acids as a component of the lipid in lieu of the saturated or unsaturated hydrocarbon fatty acids found in lipids of plant or animal origin may be used. Suspending and/or viscosity-increasing agents that may be used with emulsions include, but are not limited to, acacia, agar, alginic acid, aluminum mono-stearate, bentonite, magma, carbomer 934P, carboxymethylcellulose, calcium and sodium and sodium 12, carrageenan, cellulose, dextrin, gelatin, guar gum, hydroxyethyl cellulose, hydroxypropyl methylcellulose, magnesium aluminum silicate, methylcellulose, pectin, polyethylene oxide, polyvinyl alcohol, povidone, propylene glycol alginate, silicon dioxide, sodium alginate, tragacanth, and xanthum gum.

As described herein, lipid-encapsulated particles of the invention incorporate therapeutic agents (e.g. drugs, prodrugs, genetic materials, radioactive isotopes, or combinations thereof) in their native form or derivatized with hydrophobic or charged moieties to enhance incorporation or adsorption to the particle. The therapeutic agent may be a prodrug, including the prodrugs described, for example, by Sinkyla et al. (1975) J. Pharm. Sci. 64:181-210, Koning et al. (1999) Br. J. Cancer 80:1718-1725, U.S. Pat. No. 6,090,800 and U.S. Pat. No. 6,028,066.

The particular therapeutic agent(s) in thenongaseous, lipid-encapsulated particles of the invention is selected as appropriate for use in prophylactic measures or in treating a diagnosed disease or condition. In some embodiments, the therapeutic agents are incorporated within the core of the lipid-encapsulated particles. Such therapeutic agents for use in the methods of the invention may also include, but are not limited to antineoplastic agents, radiopharmaceuticals, nucleic acids, protein and nonprotein natural products or analogues/mimetics thereof including hormones, analgesics, muscle relaxants, narcotic agonists, narcotic agonist-antagonists, narcotic antagonists, nonsteroidal anti-inflammatories, anesthetic and sedatives, neuromuscular blockers, cytokines, antimicrobials, anti-helmintics, antimalarials, antiparasitic agents, antiviral agents, antiherpetic agents, antihypertensives, antidiabetic agents, gout related medicants, antihistamines, antiulcer medicants, anticoagulants and blood products.

In some cases, the therapeutic agent may be linked to certain proteins or peptides that can efficiently translocate across the cell membrane. Such translocatory proteins or peptides are able to mediate intercellular and/or intracellular delivery of therapeutic agents, e.g., peptides or proteins, to which they are fused. Examples of such translocatory proteins or peptides are known in the art and include, but are not limited to, human immunodeficiency virus Tat peptide and Tat-like peptides, herpes simplex virus VP22 peptide and Drosophila antennapedia protein. The intercellular transfer function generally resides in short peptides of highly basic amino acid residues termed protein transduction domains (PTD). See, for example, Fawell et al. (1994) Proc. Natl. Acad. Sci. USA 91:664-668; Elliott et al. (1997) Cell 88:223-233; Leifert et al. (2003) Mol. Ther. 8:13-20.

Genetic material, includes, for example, nucleic acids, RNA and DNA, of either natural or synthetic origin, including recombinant RNA and DNA and antisense RNA and DNA; hammerhead RNA, ribozymes, hammerhead ribozymes, antigene nucleic acids, both single and double stranded RNA and DNA and analogs thereof, immunostimulatory nucleic acid, ribooligonucleotides, antisense ribooligonucleotides, deoxyribooligonucleotides, and antisense deoxyribooligonucleotides. Other types of genetic material that may be used include, for example, genes carried on expression vectors such as plasmids, phagemids, cosmids, yeast artificial chromosomes, and defective or “helper” viruses, antigene nucleic acids, both single and double stranded RNA and DNA and analogs thereof, such as phosphorothioate and phosphorodithioate oligodeoxynucleotides. Additionally, the genetic material may be combined, for example, with proteins or other polymers.

Further description of additional therapeutic agents appropriate for use is provided herein, in particular, later in this Lipid-Encapsulated Particle Compositions section.

As described herein, the lipid-encapsulated particles may incorporate on the particle paramagnetic or super paramagnetic elements including but not limited to gadolinium, magnesium, iron, manganese in their native or in a chemically complexed form. Similarly, radioactive nuclides including positron-emitters, gamma-emitters, beta-emitters, alpha-emitters in their native or chemically-complexed form may be included on or in the particles. In some instances, adding of these moieties may permit the additional use of other clinical imaging modalities such as magnetic resonance imaging, positron emission tomography, and nuclear medicine imaging techniques in conjunction with ultrasonic imaging.

In addition, optical imaging, which refers to the production of visible representations of tissue or regions of a patient produced by irradiating those tissues or regions of a patient with electromagnetic energy in the spectral range between ultraviolet and infrared, and analyzing either the reflected, scattered, absorbed and/or fluorescent energy produced as a result of the irradiation, may be combined with the ultrasonic imaging of targeted emulsions. Examples of optical imaging include, but are not limited to, visible photography and variations thereof, ultraviolet images, infrared images, fluorimetry, holography, visible microscopy, fluorescent microscopy, spectrophotometry, spectroscopy, fluorescence polarization and the like.

Photoactive agents, i.e. compounds or materials that are active in light or that responds to light, including, for example, chromophores (e.g., materials that absorb light at a given wavelength), fluorophores (e.g., materials that emit light at a given wavelength), photosensitizers (e.g., materials that can cause necrosis of tissue and/or cell death in vitro and/or in vivo), fluorescent materials, phosphorescent materials and the like, that may be used in diagnostic or therapeutic applications. “Light” refers to all sources of light including the ultraviolet (UV) region, the visible region and/or the infrared (IR) region of the spectrum. Suitable photoactive agents that may be used in the present invention have been described by others (for example, U.S. Pat. No. 6,123,923). Further description of additional photoactive agents appropriate for use is provided herein, in particular, later in this Lipid-Encapsulated Particle Compositions section.

In addition, certain ligands, such as, for example, antibodies, peptide fragments, or mimetics of a biologically active ligand may contribute to the inherent therapeutic effects, either as an antagonistic or agonistic, when bound to specific epitopes. As an example, antibody against αᵥβ₃ integrin on neovascular endothelial cells has been shown to transiently inhibit growth and metastasis of solid tumors. The efficacy of therapeutic emulsion particles directed to the αᵥβ₃ integrin may result from the improved antagonistic action of the targeting ligand in addition to the effect of the therapeutic agents incorporated and delivered by particle itself.

In addition to that described elsewhere herein, following is further description of the various kinds of antibodies appropriate for use as targeting ligands in and/or with the lipid-encapsulated particles of the invention.

Bivalent F(ab′)₂ and monovalent F(ab) fragments can be used as ligands and these are derived from selective cleavage of the whole antibody by pepsin or papain digestion, respectively. Antibodies can be fragmented using conventional techniques and the fragments (including “Fab” fragments) screened for utility in the same manner as described above for whole antibodies. The “Fab” region refers to those portions of the heavy and light chains which are roughly equivalent, or analogous, to the sequences which comprise the branch portion of the heavy and light chains, and which have been shown to exhibit immunological binding to a specified antigen, but which lack the effector Fc portion. “Fab” includes aggregates of one heavy and one light chain (commonly known as Fab′), as well as tetramers containing the 2H and 2L chains (referred to as F(ab)₂), which are capable of selectively reacting with a designated antigen or antigen family. Methods of producing Fab fragments of antibodies are known within the art and include, for example, proteolysis, and synthesis by recombinant techniques. For example, F(ab′)₂ fragments can be generated by treating antibody with pepsin. The resulting F(ab′)₂ fragment can be treated to reduce disulfide bridges to produce Fab′ fragments. “Fab” antibodies may be divided into subsets analogous to those described herein, i.e., “hybrid Fab”, “chimeric Fab”, and “altered Fab”. Elimination of the Fc region greatly diminishes the immunogenicity of the molecule, diminishes nonspecific liver uptake secondary to bound carbohydrate, and reduces complement activation and resultant antibody-dependent cellular toxicity. Complement fixation and associated cellular cytotoxicity can be detrimental when the targeted site must be preserved or beneficial when recruitment of host killer cells and target-cell destruction is desired (e.g. anti-tumor agents).

Most monoclonal antibodies are of murine origin and are inherently immunogenic to varying extents in other species. Humanization of murine antibodies through genetic engineering has lead to development of chimeric ligands with improved biocompatibility and longer circulatory half-lives. Antibodies used in the invention include those that have been humanized or made more compatible with the individual to which they will be administered. In some cases, the binding affinity of recombinant antibodies to targeted molecular epitopes can be improved with selective site-directed mutagenesis of the binding idiotype. Methods and techniques for such genetic engineering of antibody molecules are known in the art. By “humanized” is meant alteration of the amino acid sequence of an antibody so that fewer antibodies and/or immune responses are elicited against the humanized antibody when it is administered to a human. For the use of the antibody in a mammal other than a human, an antibody may be converted to that species format.

Phage display techniques may be used to produce recombinant human monoclonal antibody fragments against a large range of different antigens without involving antibody-producing animals. In general, cloning creates large genetic libraries of corresponding DNA (cDNA) chains deducted and synthesized by means of the enzyme “reverse transcriptase” from total messenger RNA (mRNA) of human B lymphocytes. By way of example, immunoglobulin cDNA chains are amplified by polymerase chain reaction (PCR) and light and heavy chains specific for a given antigen are introduced into a phagemid vector. Transfection of this phagemid vector into the appropriate bacteria results in the expression of an scFv immunoglobulin molecule on the surface of the bacteriophage. Bacteriophages expressing specific immunoglobulin are selected by repeated immunoadsorption/phage multiplication cycles against desired antigens (e.g., proteins, peptides, nuclear acids, and sugars). Bacteriophages strictly specific to the target antigen are introduced into an appropriate vector, (e.g., Escherichia coli, yeast, cells) and amplified by fermentation to produce large amounts of human antibody fragments, generally with structures very similar to natural antibodies. Phage display techniques are known in the art and have permitted the production of unique ligands for targeting and therapeutic applications.

Polyclonal antibodies against selected antigens may be readily generated by one of ordinary skill in the art from a variety of warm-blooded animals such as horses, cows, various fowl, rabbits, mice, or rats. In some cases, human polyclonal antibodies against selected antigens may be purified from human sources.

As used herein, a “single domain antibody” (dAb) is an antibody which is comprised of a V_{H} domain, which reacts immunologically with a designated antigen. A dAb does not contain a V_{L} domain, but may contain other antigen binding domains known to exist in antibodies, for example, the kappa and lambda domains. Methods for preparing dAbs are known in the art. See, for example, Ward et al. (1989) Nature 341:544-546. Antibodies may also be comprised of V_{H} and V_{L} domains, as well as other known antigen binding domains. Examples of these types of antibodies and methods for their preparation are known in the art (see, e.g., U.S. Pat. No. 4,816,467).

Further exemplary antibodies include “univalent antibodies”, which are aggregates comprised of a heavy chain/light chain dimer bound to the Fc (i.e., constant) region of a second heavy chain. This type of antibody generally escapes antigenic modulation. See, e.g., Glennie et al. (1982) Nature 295:712-714.

“Hybrid antibodies” are antibodies wherein one pair of heavy and light chains is homologous to those in a first antibody, while the other pair of heavy and light chains is homologous to those in a different second antibody. Typically, each of these two pairs will bind different epitopes, particularly on different antigens. This results in the property of “divalence”, i.e., the ability to bind two antigens simultaneously. Such hybrids may also be formed using chimeric chains, as set forth herein.

The invention also encompasses “altered antibodies”, which refers to antibodies in which the naturally occurring amino acid sequence in a vertebrate antibody has been varied. Utilizing recombinant DNA techniques, antibodies can be redesigned to obtain desired characteristics. The possible variations are many, and range from the changing of one or more amino acids to the complete redesign of a region, for example, the constant region. Changes in the variable region may be made to alter antigen binding characteristics. The antibody may also be engineered to aid the specific delivery of an emulsion to a specific cell or tissue site. The desired alterations may be made by known techniques in molecular biology, e.g., recombinant techniques, site directed mutagenesis, and other techniques.

“Chimeric antibodies”, are antibodies in which the heavy and/or light chains are fusion proteins. Typically the constant domain of the chains is from one particular species and/or class, and the variable domains are from a different species and/or class. The invention includes chimeric antibody derivatives, i.e., antibody molecules that combine a non-human animal variable region and a human constant region. Chimeric antibody molecules can include, for example, the antigen binding domain from an antibody of a mouse, rat, or other species, with human constant regions. A variety of approaches for making chimeric antibodies have been described and can be used to make chimeric antibodies containing the immunoglobulin variable region which recognizes selected antigens on the surface of targeted cells and/or tissues. See, for example, Morrison et al. (1985) Proc. Natl. Acad. Sci. U.S.A. 81:6851; Takeda et al. (1985) Nature 314:452; U.S. Pat. Nos. 4,816,567 and 4,816,397; European Patent Publications EP171496 and EP173494; United Kingdom patent GB 2177096B.

Bispecific antibodies may contain a variable region of an anti-target site antibody and a variable region specific for at least one antigen on the surface of the lipid-encapsulated emulsion. In other cases, bispecific antibodies may contain a variable region of an anti-target site antibody and a variable region specific for a linker molecule. Bispecific antibodies may be obtained forming hybrid hybridomas, for example by somatic hybridization. Hybrid hybridomas may be prepared using the procedures known in the art such as those disclosed in Staerz et al. (1986, Proc. Natl. Acad. Sci. U.S.A. 83:1453) and Staerz et al. (1986, Immunology Today 7:241). Somatic hybridization includes fusion of two established hybridomas generating a quadroma (Milstein et al. (1983) Nature 305:537-540) or fusion of one established hybridoma with lymphocytes derived from a mouse immunized with a second antigen generating a trioma (Nolan et al. (1990) Biochem. Biophys. Acta 1040:1-11). Hybrid hybridomas are selected by making each hybridoma cell line resistant to a specific drug-resistant marker (De Lau et al. (1989) J. Immunol. Methods 117:1-8), or by labeling each hybridoma with a different fluorochrome and sorting out the heterofluorescent cells (Karawajew et al. (1987) J. Immunol. Methods 96:265-270).

Bispecific antibodies may also be constructed by chemical means using procedures such as those described by Staerz et al. (1985) Nature 314:628 and Perez et al. (1985) Nature 316:354. Chemical conjugation may be based, for example, on the use of homo- and heterobifunctional reagents with E-amino groups or hinge region thiol groups. Homobifunctional reagents such as 5,5′-dithiobis(2-nitrobenzoic acid) (DNTB) generate disulfide bonds between the two Fabs, and O-phenylenedimaleimide (O-PDM) generate thioether bonds between the two Fabs (Brenner et al. (1985) Cell 40:183-190, Glennie et al. (1987) J. Immunol. 139:2367-2375). Heterobifunctional reagents such as N-succinimidyl-3-(2-pyridylditio)propionate (SPDP) combine exposed amino groups of antibodies and Fab fragments, regardless of class or isotype (Van Dijk et al. (1989) Int. J. Cancer 44:738-743).

Bifunctional antibodies may also be prepared by genetic engineering techniques. Genetic engineering involves the use of recombinant DNA based technology to ligate sequences of DNA encoding specific fragments of antibodies into plasmids, and expressing the recombinant protein. Bispecific antibodies can also be made as a single covalent structure by combining two single chains Fv (scFv) fragments using linkers (Winter et al. (1991) Nature 349:293-299); as leucine zippers coexpressing sequences derived from the transcription factors fos and jun (Kostelny et al. (1992) J. Immunol. 148:1547-1553); as helix-turn-helix coexpressing an interaction domain of p53 (Rheinnecker et al. (1996) J. Immunol. 157:2989-2997), or as diabodies (Holliger et al. (1993) Proc. Natl. Acad. Sci. U.S.A. 90:6444-6448).

In addition to that described elsewhere herein, following is further description of coupling agents appropriate for use in coupling a primer material, for example, to a specific binding or targeting ligand. Additional coupling agents use a carbodiimide such as 1-ethyl-3-(3-N,N dimethylaminopropyl)carbodiimide hydrochloride or 1-cyclohexyl-3-(2-morpholinoethyl)carbodiimide methyl-p-toluenesulfonate. Other suitable coupling agents include aldehyde coupling agents having either ethylenic unsaturation such as acrolein, methacrolein, or 2-butenal, or having a plurality of aldehyde groups such as glutaraldehyde, propanedial or butanedial. Other coupling agents include 2-iminothiolane hydrochloride, bifunctional N-hydroxysuccinimide esters such as disuccinimidyl substrate, disuccinimidyl tartrate, bis[2-(succinimidooxycarbonyloxy)ethyl]sulfone, disuccinimidyl propionate, ethylene glycolbis(succinimidyl succinate); heterobifunctional reagents such as N-(5-azido-2-nitrobenzoyloxy)succinimide, p-azidophenylbromide, p-azidophenylglyoxal, 4-fluoro-3-nitrophenylazide, N-hydroxysuccinimidyl-4-azidobenzoate, m-maleimidobenzoyl N-hydroxysuccinimide ester, methyl-4-azidophenylglyoxal, 4-fluoro-3-nitrophenyl azide, N-hydroxysuccinimidyl-4-azidobenzoate hydrochloride, p-nitrophenyl 2-diazo-3,3,3 trifluoropropionate, N-succinimidyl-6-(4′-azido-2′-nitrophenylamino)hexanoate, succinimidyl 4-(N-maleimidomethyl)cyclohexane-1-carboxylate, succinimidyl 4-(p-maleimidophenyl)butyrate, N-succinimidyl(4-azidophenyldithio)propionate, N-succinimidyl 3-(2-pyridyldithio)propionate, N-(4-azidophenylthio)phthalamide; homobifunctional reagents such as 1,5-difluoro-2,4-dinitrobenzene, 4,4′-difluoro-3,3′-dinitrodiphenylsulfone, 4,4′-diisothiocyano-2,2′-disulfonic acid stilbene, p-phenylenediisothiocyanate, carbonylbis(L-methionine p-nitrophenyl ester), 4,4′-dithiobisphenylazide, erythritolbiscarbonate and bifunctional imidoesters such as dimethyl adipimidate hydrochloride, dimethyl suberimidate, dimethyl 3,3′-dithiobispropionimidate hydrochloride and the like.

In addition to that described elsewhere herein, following is further description of therapeutic agents that may be incorporated onto and/or within the nanoparticles of the invention. Generally, the therapeutic agents can be derivatized with a lipid anchor to make the agent lipid soluble or to increase its solubility in lipid, therefor increasing retension of the agent in the lipid layer of the emulsion and/or in the lipid membrane of the target cell. Such therapeutic emulsions may also include, but are not limited to antineoplastic agents, including platinum compounds (e.g., spiroplatin, cisplatin, and carboplatin), methotrexate, fluorouracil, adriamycin, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, mitotane, procarbazine hydrochloride dactinomycin (actinomycin D), daunorubicin hydrochloride, doxorubicin hydrochloride, taxol, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, interferon α-2a, interferon α-2b, teniposide (VM-26), vinblastine sulfate (VLB), vincristine sulfate, bleomycin, bleomycin sulfate, methotrexate, adriamycin, arabinosyl, hydroxyurea, procarbazine, dacarbazine, mitotic inhibitors such as etoposide and other vinca alkaloids; radiopharmaceuticals such as but not limited to radioactive iodine, samarium, strontium cobalt, yittrium and the like; protein and nonprotein natural products or analogues/mimetics thereof including hormones such as but not limited to growth hormone, somatostatin, prolactin, thyroid, steroids, androgens, progestins, estrogens and antiestrogens; analgesics including but not limited to antirheumatics, such as auranofin, methotrexate, azathioprine, sulfazalazine, leflunomide, hydrochloroquine, and etanercept; muscle relaxants such as baclofen, dantrolene, carisoprodol, diazepam, metaxalone, cyclobenzaprine, chlorzoxazone, tizanidine; narcotic agonists such as codeine, fentanyl, hydromorphone, Ileavorphanol, meperidine, methadone, morphine, oxycodone, oxymorphone, propoxyphene; narcotic agonist-antagonists such as buprenorphine, butorphanol, dezocine, nalbuphine, pentazocine; narcotic antagonists such as nalmefene and naloxone, other analgesics including ASA, acetominophen, tramadol, or combinations thereof; nonsteroidal anti-inflammatories including but not limited to celecoxib, diclofenac, diflunisal, etodolac, fenoprofen, flurbiprofen, ibuprofen, indomethacin, ketoprofen, ketorolac, naproxen, oxaproxen, rofecoxib, salisalate, suldindac, tolmetin; anesthetic and sedatives such as etomidate, fentanyl, ketamine, methohexital, propofol, sufentanil, thiopental, and the like; neuromuscular blockers such as but not limited to pancuronium, atracurium, cisatracurium, rocuronium, succinylcholine, vercuronium; antimicrobials including aminoglycosides, antifungal agents including amphotericin B, clotrimazole, fluconazole, flucytosine, griseofulvin, itraconazole, ketoconazole, nystatin, and terbinafine; anti-helmintics; antimalarials, such as chloroquine, doxycycline, mefloquine, primaquine, quinine; antimycobacterial including dapsone, ethambutol, ethionamide, isoniazid, pyrazinamide, rifabutin, rifampin, rifapentine; antiparasitic agents including albendazole, atovaquone, iodoquinol, ivermectin, mebendazole, metronidazole, pentamidine, praziquantel, pyrantel, pyrimethamine, thiabendazole; antiviral agents including abacavir, didanosine, lamivudine, stavudine, zalcitabine, zidovudine as well as protease inhibitors such as indinavir and related compounds, anti-CMV agents including but not limited to cidofovir, foscarnet, and ganciclovir; antiherpetic agents including amatadine, rimantadine, zanamivir; interferons, ribavirin, rebetron; carbapenems, cephalosporins, fluoroquinones, macrolides, penicillins, sulfonamides, tetracyclines, and other antimicrobials including aztreonam, chloramphenieol, fosfomycin, furazolidone, nalidixic acid, nitrofurantoin, vancomycin and the like; nitrates, antihypertensives including diuretics, beta blockers, calcium channel blockers, angiotensin converting enzyme inhibitors, angiotensin receptor antagonists, antiadrenergic agents, anti-dysrhythmics, antihyperlipidemic agents, antiplatelet compounds, pressors, thrombolytics, acne preparations, antipsoriatics; corticosteroids; androgens, anabolic steroids, bisphosphonates; sulfonoureas and other antidiabetic agents; gout related medicants; antihistamines, antitussive, decongestants, and expectorants; antiulcer medicants including antacids, 5-HT receptor antagonists, H2-antagonists, bismuth compounds, proton pump inhibitors, laxatives, octreotide and its analogues/mimetics; anticoagulants; immunization antigens, immunoglobins, immunosuppressive agents; anticonvulsants, 5-HT receptor agonists, other migraine therapies; parkinsonian agents including anticholinergics, and dopaminergics; estrogens, GnRH agonists, progestins, estrogen receptor modulators, tocolytics, uterotnics, thyroid agents such as iodine products and anti-thyroid agents; blood products such as parenteral iron, hemin, hematoporphyrins and their derivatives.

In addition to that described elsewhere herein, following is further description of additional photoactive agents appropriate for use in optical imaging of the nanoparticles of the invention. Suitable photoactive agents include but are not limited to, for example, fluoresceins, indocyanine green, rhodamine, triphenylmethines, polymethines, cyanines, fullerenes, oxatellurazoles, verdins, rhodins, perphycenes, sapphyrins, rubyrins, cholesteryl 4,4-difluoro-5,7-dimethyl-4-bora-3a,4a-diaza-s-indacene-3-dodecanoate, cholesteryl 12-(N-methyl-N-(7-nitrobenz-2-oxa-1,3-diazol-4-yl)amino)dodecanate, cholesteryl cis-parinarate, cholesteryl 3-((6-phenyl)-1,3,5-hexatrienyl)phenyl-proprionate, cholesteryl 1-pyrenebutyrate, cholesteryl-1-pyrenedecanoate, cholesteryl 1-pyrenehexanoate, 22-(N-(7-nitrobenz-2-oxa-1,3-diazol-4-yl)amino)-23,24-bisnor-5-cholen-3β-ol, 22-(N-(7-nitrobenz-2-oxa-1,3-diazol-4-yl)amino)-23,24-bisnor-5-cholen-3β-yl cis-9-octadecenoate, 1-pyrenemethyl3-hydroxy-22,23-bisnor-5-cholenate, 1-pyrene-methyl 3β-(cis-9-octadecenoyloxy)-22,23-bisnor-5-cholenate, acridine orange 10-dodecyl bromide, acridine orange 10-nonyl bromide, 4-(N,N-dimethyl-N-tetradecylammonium)-methyl-7-hydroxycoumarin)chloride, 5-dodecanoylaminofluorescein, 5-dodecanoylaminofluorescein-bis-4,5-dimethoxy-2-nitrobenzyl ether, 2-dodecylresorufin, fluorescein octadecyl ester, 4-heptadecyl-7-hydroxycoumarin, 5-hexadecanoylaminoeosin, 5-hexadecanoylaminofluorescein, 5-octadecanoylaminofluorescein, N-octadecyl-N′-(5-(fluoresceinyl))thiourea, octadecyl rhodamine B chloride, 2-(3-(diphenylhexatrienyl)-propanoyl)-1-hexadecanoyl-sn-glycero-3-phosphocholine, 6-N-(7-nitrobenz-2-oxa-1,3-diazol-4-yl)amino)hexanoic acid, 1-hexadecanoyl-2-(1-pyrenedecanoyl)-sn-glycero-3-phosphocholine, 1,1′-dioctadecyl-3,3,3′,3′-tetramethyl-indocarbocyanine perchlorate, 12-(9-anthroyloxy)oleic acid, 5-butyl-4,4-difluoro-4-bora-3a,4a-diaza-s-indacene-3-nonanoic acid, N-(Lissamine™ rhodamine B sulfonyl)-1,2-dihexadecanoyl-sn-glycero-3-phosphoethanolamine, triethylammonium salt, phenylglyoxal monohydrate, naphthalene-2,3-dicarboxaldehyde, 8-bromomethyl-4,4-difluoro-1,3,5,7-tetramethyl-4-bora-3a,4a-diaza-s-indacene, o-phthaldialdehyde, Lissamine™ rhodamine B sulfonyl chloride, 2′,7′-difluorofluorescein, 9-anthronitrile, 1-pyrenesulfonyl chloride, 4-(4-(dihexadecylamino)-styryl)-N-methylpyridinium iodide, chlorins, such as chlorin, chlorin e6, bonellin, mono-L-aspartyl chlorin e6, mesochlorin, mesotetraphenylisobacteriochlorin, and mesotetraphenylbacteriochlorin, hypocrellin B, purpurins, such as octaethylpurpurin, zinc(II) etiopurpurin, tin(IV) etiopurpurin and tin ethyl etiopurpurin, lutetium texaphyrin, photofrin, metalloporphyrins, protoporphyrin IX, tin protoporphyrin, benzoporphyrin, haematoporphyrin, phthalocyanines, naphthocyanines, merocyanines, lanthanide complexes, silicon phthalocyanine, zinc phthalocyanine, aluminum phthalocyanine, Ge octabutyoxyphthalocyanines, methyl pheophorbide-α-(hexyl-ether), porphycenes, ketochlorins, sulfonated tetraphenylporphines, δ-aminolevulinic acid, texaphyrins, including, for example, 1,2-dinitro-4-hydroxy-5-methoxybenzene, 1,2-dinitro-4-(1-hydroxyhexyl)oxy-5-methoxybenzene, 4-(1-hydroxyhexyl)oxy-5-methoxy-1,2-phenylenediamine, and texaphyrin-metal chelates, including the metals Y(III), Mn(II), Mn(III), Fe(II), Fe(III) and the lanthanide metals Gd(III), Dy(III), Eu(III), La(III), Lu(III) and Tb(III), chlorophyll, carotenoids, flavonoids, bilins, phytochromes, phycobilins, phycoerythrins, phycocyanines, retinoic acids, retinoins, retinates, or combinations of any of the above.

One skilled in the art will readily recognize or can readily determine which of the above compounds are, for example, fluorescent materials and/or photosensitizers. LISSAMINE is the trademark for N-ethyl-N-[4-[[4-[ethyl [(3-sulfophenyl)methyl]amino]phenyl](4-sulfopheny-1)-methylene]-2,5-cyclohexadien-1-ylidene]-3-sulfobenzene-methanaminium hydroxide, inner salt, disodium salt and/or ethyl[4[p[ethyl(m-sulfobenzyl)amino]-α-(p-sulfophenyl)benzylidene]-2,5-cyclohexadien-1-ylidene](m-sulfobenzyl)ammonium hydroxide inner salt disodium salt (commercially available from Molecular Probes, Inc., Eugene, Oreg.). Other suitable photoactive agents for use in the present invention include those described in U.S. Pat. No. 4,935,498, such as a dysprosium complex of 4,5,9,24-tetraethyl-16-(1-hydroxyhexyl)oxy-17 methoxypentaazapentacyclo-(20.2.1.1³,6.1⁸,11.0¹⁴, 9)-heptacosa-1,3,5,7,9,11(27),12,14,16,18,20,22(25),23-tridecaene and dysprosium complex of 2-cyanoethyl-N,N-diisopropyl-6-(4,5,9,24-tetraethyl-17-methoxypentaazapentacyclo-(20.2.1.1³,6.1⁸,11.0¹⁴,19)-heptacosa-1,3,5,7,9,11(27), 12,14,16,18,20,22(25),23-tridecaene-16-(1-oxy)hexylphosphoramidite.

Methods of Preparation of the Lipid-Encapsuated Particles

The lipid-encapsulated particles of the present invention may be prepared by various techniques. Typically, lipid membranes of a lipid-encapsulated particle are made artificially from phospholipids, glycolipids, lipids, steroids such as cholesterol, related molecules, or a combination thereof by any technique known in the art, including but not limited to sonication, extrusion, or removal of detergent from lipid-detergent complexes. For example, in a typical procedure for preparing perfluorocarbon based nanoparticles, the perfluorocarbon and the components of the lipid/surfactant coating are fluidized in aqueous medium to form an emulsion. The functional components of the surface layer may be included in the original emulsion, or may later be covalently coupled to the surface layer subsequent to the formation of the nanoparticle emulsion. In one particular instance, for example, where a nucleic acid targeting agent or therapeutic agent is to be included, the coating may employ a cationic surfactant and the nucleic acid adsorbed to the surface after the particle is formed.

Generally, the emulsifying process involves directing high pressure streams of mixtures containing the aqueous solution, a primer material or the specific binding species, the oil, e.g., a perfluorocarbon, and a surfactant (if any) so that they impact one another to produce emulsions of narrow particle size and distribution. The MICROFLUIDIZER apparatus (Microfluidics, Newton, Mass.) can be used to make the preferred emulsions. The apparatus is also useful to post-process emulsions made by sonication or other conventional methods. Feeding a stream of emulsion droplets through the MICROFLUIDIZER apparatus yields formulations small size and narrow particle size distribution.

An alternative method for making the emulsions involves sonication of a mixture of an oil, e.g., a perfluorocarbon, and an aqueous solution containing a suitable primer material and/or specific binding species. Generally, these mixtures include a surfactant. Cooling the mixture being emulsified, minimizing the concentration of surfactant, and buffering with a saline buffer will typically maximize both retention of specific binding properties and the coupling capacity of the primer material. These techniques provide excellent emulsions with high activity per unit of absorbed primer material or specific binding species.

Phospholipids may be obtained from natural sources, such as egg or soybean phosphatidylcholine, brain phosphatidic acid, brain or plant phosphatidylinositol, heart cardiolipin, or plant or bacterial phosphatidylethanolamine. Phospholipids for use in encapsulation compositions of the invention are either purchased from chemical suppliers or synthesized using techniques known to those of skill in the art.

When high concentrations of a primer material or target binding species coated on lipid emulsions, the mixture should generally be heated during sonication and have a relatively low ionic strength and moderate to low pH. Too low an ionic strength, too low a pH or too much heat may cause some degradation or loss of all of the useful binding properties of the specific binding species or the coupling capacity of the primer material. Careful control and variation of the emulsification conditions can optimize the properties of the primer material or the specific binding species while obtaining high concentrations of coating.

The emulsion particle sizes can be controlled and varied by modification of the emulsification techniques and the chemical components. Techniques and equipment for determining particle sizes are known in the art and include, but not limited to, laser light scattering and an analyzer for determining laser light scattering by particles.

In some cases, the lipid-encapsulated particles typically contain hundreds or thousands of molecules of the therapeutic agent, targeting ligand, and/or radionuclide. The number of targeting agents per particle is typically of the order of several hundred while the particle may also contain variable concentrations of therapeutic agents, fluorophores, and/or radionuclides.

In addition to the inclusion of biologically active materials for delivery, the inclusion of radionuclides makes the particles and methods of the invention useful further useful as therapeutic for radiation treatment or as diagnostic for imaging. The particles need not contain an ancillary agent since, in some cases, the particles are particularly useful themselves as ultrasound contrast agents. Other imaging agents include fluorophores, such as fluorescein or dansyl. A multiplicity of such activities may be included; thus, images can be obtained of targeted tissues at the same time active substances are delivered to them.

Processess for preparing liposomes are known in the art. The lipid vesicles can be prepared by any suitable technique known in the art. Methods include, but are not limited to, microencapsulation, microfluidization, LLC method, ethanol injection, freon injection, detergent dialysis, hydration, sonication, and reverse-phase evaporation. Reviewed, for example, in Watwe et al. (1995) Curr. Sci. 68:715-724. Techniques may be combined in order to provide vesicles with the most desirable attributes. Generally, the size of the liposome depends on the method chosen. Depending on the choice of method, the resulting liposomes will have various abilities to entrap aqueous material and differ in their space-to-lipid ratios.

For example, liposomes may be prepared by mixing the phospholipid and other components which form part of the structure of the liposome in an organic solvent, evaporating off the solvent, resuspending in aqueous solvent, and finally lyophilizing the lipid/phospholipid composition. The lyophilized composition is then reconstituted in a buffer containing the substance to be encapsulated.

In another method, the liposomes are prepared by mixing the lipids to be used in the desired proportion in a container such as a glass pear-shaped flask having a volume ten times greater than the anticipated suspension of liposomes. Using a rotary evaporator, the solvent is removed at approximately 40° C. under negative pressure. The composition may then be dried further in a desiccator under vacuum, and is stable for about one week. The dried lipids may be rehydrated at approximately 30 mM phospholipid in sterile, pyrogen-free water by shaking until all lipid film is off the glass. The aqueous liposomes can then be separated in aliquots, lyophilized and sealed under vacuum.

Alternatively, liposomes can be prepared according to the methods described in Bangham et al. (1965) J. Mol. Biol. 13: 238-252, Gregoriadis in Drug Carriers in Biology and Medicine, G. Gregoriadis, Ed. (1979) pp. 287-341; Szoka et al. (1978) Proc Natl Acad Sci USA 75: 4194-4198.

Liposomes may also be prepared with surface stabilizing hydrophilic polymer-lipid conjugates such as polyethylene glycol-distearoylphosphatidylethanolamine (PEG-DSPE), to enhance circulation longevity. The incorporation of negatively charged lipids such as phosphatidylglycerol (PG) and phosphatidylinositol (PI) may also be added to liposome formulations to increase the circulation longevity of the carrier. These lipids may be employed to replace hydrophilic polymer-lipid conjugates as surface stabilizing agents. Embodiments of this invention may make use of cholesterol-free liposomes containing PG or PI to prevent aggregation thereby increasing the blood residence time of the complexes.

A stabilizing agent can be included in the compositions either by adding the appropriate proportion of the stabilizing agent in the preparation of a lyophilized lipid mixture, or by adding the stabilizing agent to the reconstitution buffer. The stabilizing agent can be added as a single detergent or can, of course, be added as a mixture of appropriate detergents. The stabilizing agent can be a nonionic detergent with appropriate physical characteristics. Specifically, the nonionic detergent must be soluble at a temperature that does not adversely affect the integrity of the liposomes and that does not denature or otherwise interfere with the ability of the targeting ligand to bind to the target cell. For example, the detergent must be soluble at a biologically reasonable temperature.

The proportion of the stabilizing agent to be included in the original phospholipid/lipid mixture or the concentration of the stabilizing agent in the reconstituting buffer will depend on the nature of the substance to be encapsulated and can be optimized using routine experimentation. In some embodiments, the stabilizing agent will be present at about 0.2-5 mole % based on the liposomal lipid mixture.

The invention involves targeting ligand bearing, lipid-encapsulated particles which have loaded with a therapeutic agent of use in delivery of the agent to the target. As used herein, the term “loading” refers to introducing into or onto a lipid-encapsulated particle at least one therapeutic agent. In one embodiment, the agent is loaded by becoming internalized into the lipid-encapsulated particle. In another embodiment, the agent is loaded by becoming coupled onto the surface of the lipid-encapsulated particle and/or embedded in the lipid coating the lipid-encapsulated particle. Loading of an lipid-encapsulated particle with more than one agent may be performed such that the agents are loaded individually (in sequence) or together (simultaneously or concurrently). Loading can occur before, during and/or after the targeting ligand is coupled to the surface of the lipid-encapsulated particle. Loading can be performed in a procedure separate from the procedure coupling a targeting ligand to the surface of the lipid-encapsulated particle or, in some cases, the procedures can be concurrent. Agents may be first admixed at the time of contact with the lipid-encapsulated particles or prior to that time.

Loading may be performed by a procedure known in the art, the particular technique used is dependent on the nature of the lipid-encapsulated particle and the agent(s). For example, agents may be loaded into liposomes using both passive and active methods. It will be appreciated by one skilled in the art that combinations of methods may be used to facilitate the loading of a lipid-encapsulated particle with agents of interest. Likewise, it will be appreciated that, when more than one agent is to be loaded, such as a first and second agent, the first and second agent may be loaded concurrently or sequentially, in either order, into a lipid-encapsulated particle.

Passive methods of loading agents in liposomes involve encapsulating the agent during the preparation of the liposomes. In this method, the agent may be membrane associated or encapsulated within an entrapped aqueous space. This includes a passive entrapment method described by Bangham, et al. (1965) J. Mol. Biol. 12:238, where the aqueous phase containing the agent of interest is put into contact with a film of dried vesicle-forming lipids deposited on the walls of a reaction vessel. Upon agitation by mechanical means, swelling of the lipids will occur and multilamellar vesicles (MLV) will form. Using extrusion, the MLVs can be converted to large unilamellar vesicles (LUV) or small unilamellar vesicles (SUV). Another method of passive loading that may be used includes that described by Deamer et al. (1976) Biochim. Biophys. Acta 443:629. This method involves dissolving vesicle-forming lipids in ether and, instead of first evaporating the ether to form a thin film on a surface, this film being thereafter put into contact with an aqueous phase to be encapsulated, the ether solution is directly injected into said aqueous phase and the ether is evaporated afterwards, whereby liposomes with encapsulated agents are obtained. A further method that may be employed is the Reverse Phase Evaporation (REV) method described by Szoka et al. (1978) P.N.A.S. 75:4194, in which a solution of lipids in a water insoluble organic solvent is emulsified in an aqueous carrier phase and the organic solvent is subsequently removed under reduced pressure.

Other methods of passive entrapment that may be used include subjecting liposomes to successive dehydration and rehydration treatment, or freezing and thawing. Dehydration is carried out by evaporation or freeze-drying. See, for example, Gregoriadis et al. (1987) Vaccine 5:145-151; Kirby et al., Biotechnology (1984) 979-984. Also, liposomes prepared by sonication are mixed in aqueous solution with the solute to be encapsulated, and the mixture is dried under nitrogen in a rotating flask. Upon rehydration, large liposomes are produced in which a significant fraction of the solute has been encapsulated. Shew et al. (1985) Biochim. et Biophys. Acta 816:1-8.

Passive encapsulation of two or more therapeutic agents is possible for many agent combinations. This approach is limited by the solubility of the agents in aqueous buffer solutions and the large percentage of agent that is not trapped within the delivery system. The loading may be improved by co-lyophilizing the drugs with the lipid sample and rehydrating in the minimal volume allowed to solubilize the drugs. The solubility may be improved by varying the pH of the buffer, increasing temperature or addition or removal of salts from the buffer.

Active methods of loading may also be used. For example, liposomes may be loaded according to a metal-complexation or pH gradient loading technique. With pH gradient loading, liposomes are formed which encapsulate an aqueous phase of a selected pH. Hydrated liposomes are placed in an aqueous environment of a different pH selected to remove or minimize a charge on the agent to be encapsulated. Once the agent moves inside the liposome, the pH of the interior results in a charged agent state, which prevents the agent from permeating the lipid bilayer, thereby entrapping the agent in the liposome.

To create a pH gradient, the original external medium can be replaced by a new external medium having a different concentration of protons. The replacement of the external medium can be accomplished by various techniques, such as, by passing the lipid vesicle preparation through a gel filtration column, e.g., a Sephadex G-50 column, which has been equilibrated with the new medium, or by centrifugation, dialysis, or related techniques. The internal medium may be either acidic or basic with respect to the external medium.

After establishment of a pH gradient, a pH gradient loadable agent is added to the mixture and encapsulation of the agent in the liposome occurs as described above. Loading using a pH gradient may be carried out according to methods described in U.S. Pat. Nos. 5,616,341, 5,736,155 and 5,785,987 incorporated herein by reference. Various methods known in the art may be employed to establish and maintain a pH gradient across a liposome. See, for example, U.S. Pat. Nos. 5,837,282, 5,785,987 and 5,939,096.

Two or more agents may be loaded into a liposome using the same active loading methods or may involve the use of different active loading methods. For instance, metal complexation loading may be utilized to actively load multiple agents or may be coupled with another active loading technique, such as pH gradient loading. Metal-based active loading typically uses liposomes with passively encapsulated metal ions (with or without passively loaded therapeutic agents). Various salts of metal ions are used, presuming that the salt is pharmaceutically acceptable and soluble in an aqueous solutions. Actively loaded agents are selected based on being capable of forming a complex with a metal ion and thus being retained when so complexed within the liposome, yet capable of loading into a liposome when not complexed to metal ions. Agents that are capable of coordinating with a metal typically comprise coordination sites such as amines, carbonyl groups, ethers, ketones, acyl groups, acetylenes, olefins, thiols, hydroxyl or halide groups or other suitable groups capable of donating electrons to the metal ion thereby forming a complex with the metal ion. Uptake of an agent may be established by incubation of the mixture at a suitable temperature after addition of the agent to the external medium. Depending on the composition of the liposome, temperature and pH of the internal medium, and chemical nature of the agent, uptake of the agent may occur over a time period of minutes or hours. Methods of determining whether coordination occurs between an agent and a metal within a liposome include spectrophotometric analysis and other conventional techniques well known to those of skill in the art.

Furthermore, liposome loading efficiency and retention properties using metal-based procedures carried out in the absence of an ionophore in the liposome are dependent on the metal employed and the lipid composition of the liposome. By selecting lipid composition and a metal, loading or retention properties can be tailored to achieve a desired loading or release of a selected agent from a liposome.

As used herein, an “individual” is a vertebrate, preferably a mammal, more preferably a human. Mammals include, but are not limited to, humans, farm animals, sport animals, rodents and pets.

As used herein, an “effective amount” or a “sufficient amount” of a substance is that amount sufficient to effect beneficial or desired results, including clinical results, and, as such, an “effective amount” depends upon the context in which it is being applied. An effective amount can be administered in one or more administrations.

As used herein, the singular form “a”, “an”, and “the” includes plural references unless indicated otherwise. For example, “a” target cell includes one or more target cells.

The following Examples are offered to illustrate but not to limit the invention.

### EXAMPLES

The following examples demonstrate the use of ultrasonic methods to increase intracelullar delivery of an agent. Using clinical levels of ultrasound energy with the exemplary PFC nanoparticles targeted to cells expressing the integrin αᵥβ₃, these results support the feasibility of using such nanoparticles for ultrasonically enhanced noncavitational drug delivery.

### Example 1

Nanoparticles complexed with ligands targeted to αᵥβ₃ were incubated with C32 melanoma cells which express αᵥβ₃ in culture. Control nanoparticles without a targeting ligand to αᵥβ₃ were also incubated with C32 melanoma cells. The nanoparticles contained fluorescein-conjugated phospholipid incoporated into the surfactant layer for confocal microscopic imaging of the particles and cells.

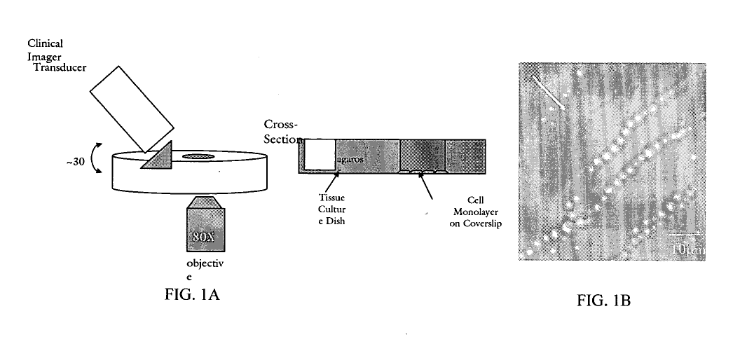

A clinical medical imager (Acuson Sequoia) was used with a broadband (2-3.5 MHz, 3Va2) phased-array transducer to apply ultrasound to cells in culture. The transducer was applied from the side at a 30-degree angle (FIG. 1A) to a modified tissue culture dish. For the modified tisuue culture dish, a hole was drilled into a tissue culture dish (polymethylpentene, Nalge) and a watertight sealant was used to secure a coverslip (Thermanox, Nunc) to the bottom of the dish. Cells were grown on the coverslip for 2 days at 37° C. to allow for attachment before exposure to the experimental conditions. A 2% agarose disk, used to couple the ultrasound to the cells, was made to fit the dish and a hole was cored out of the agarose over the coverslip. Cells were grown on the coverslip for 2 days at 37° C. to allow for attachment before exposure to the experimental conditions.

The experiments took place on top of an inverted phase-contrast microscope (Nikon Diaphot 300), which permitted simultaneous microscopic visualization of cell interactions during exposure to calibrated levels of ultrasound energy (mechanical index (MI): 1.9; exposure time: 5 minutes; 2-3 MHz phased array transducer: Acuson 3Va2). Differences between the treatment groups were evaluated for significance using analysis of variance (ANOVA) with the Statistical Analysis System (SAS, Cary, N.C.). A p-value of 0.05 was considered statistically significant.

Nanoparticle association with cells was quantified by analyzing for the presence of the perfluorocarbon (PFC) core with gas chromatography. PFC content measured by gas chromatography (Agilent, 6890 Series) was used as a tracer to quantify delivery of particles to cells. Fluorescent imaging after treatment was conducted with a confocal microscope (BioRad MRC1024), using fluorescein filter sets. Survivability, immediately (within 1 hour) and 24 hours after treatment, was determined by trypan blue exclusion. The percentage of trypan blue-positive cells in each condition (control, ultrasound alone, nanoparticles alone, and ultrasound with nanoparticles) was used to calculate cell survival. Within one hour after isonification, cell viability was greater than 98% for cells in each condition. Twenty-four hours after treatment, cell viability was about 90% for both treated and untreated cells. Thus, neither the particles nor the energy used had an effect on cell viability.

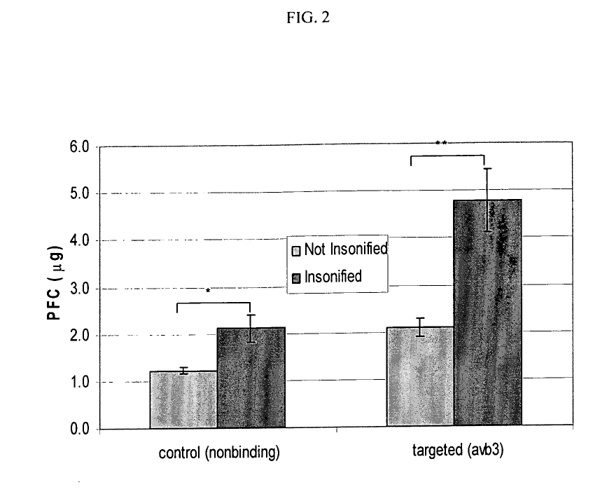

After nanoparticle binding to cells and application of ultrasound, a greater than 2-fold increase in PFC content of the targeted (αᵥβ₃) cells was observed with ultrasound than without ultrasound. As depicted in FIG. 2, 4.79+/−0.66 micrograms PFC with ultrasound as compared to 2.10+/−0.20 micrograms PFC without ultrasound (p<0.005). For control nontargeted nanoparticles, ultrasound exposure also increased PFC deposition in the cells, but the overall level was substantially less.

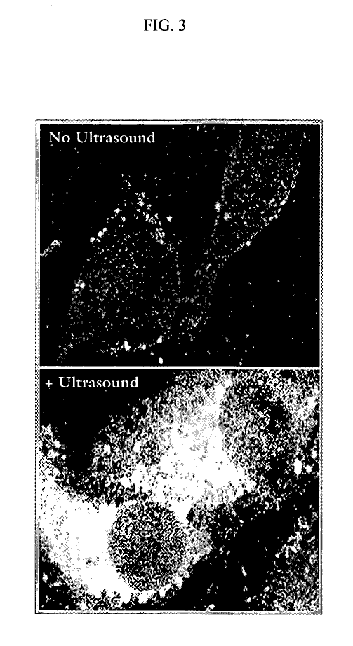

The relative amount of lipid delivered from the lipid monolayer of the nanoparticle to the cell was determined using a fluorescent lipid incorporated into the surfactant layer which was imaged with confocal microscopy. This technique allowed direct visualization of the lipid delivery occurring in the C32 cells. As shown in FIG. 3, a dramatic augmentation of lipid exchange occurs after insonification of targeted particles bound to cells, since the fluorescent signal is essentially saturated over the entire cell. In this case, for the ultrasound treated cells, the microscope diaphragm was closed to less than ⅓ its diameter as compared to the diaphragm diameter used to image cells without ultrasound treatment. Since intensity impinging on the CCD camera used to digitize the image is proportional to the area of the diaphragm that allows passage of the light, this result strongly suggests a potential augmentation in fluorescence intensity due to enhanced fluorescent lipid exchange after ultrasound treatment of at least ten times that of the untreated cells. Without being held to a particular theory, the large increase in fluorescence intensity relative to the measured increase in PFC content suggests that the predominant interaction enhanced by ultrasound application is lipid exchange and/or lipid vesicle fusion rather than intact particle uptake in endosomal compartments. Furthermore, the distribution of labeled lipid in the cell is not compartmentalized (i.e., it is diffusely distributed throughout the cell membrane and cytoplasm), also indicating a lipid exchange mechanism rather than intact particle uptake.

Videodensitometric data show that nanoparticles were not destroyed by ultrasound exposure and the alignment of nanoparticles relative to incident acoustic field demonstrate conclusively that acoustic radiation forces (primary and secondary) influence the nanoparticles and implicate these forces as participants in the enhanced delivery (see, for example, FIG. 1B). The primary radiation force causes movement of particles along the direction pointing away from the wave source and the secondary radiation force results in a repulsive force between particles whose relative orientation is parallel to the incident wave and an attractive force between particles whose relative orientation is perpendicular to the incident wave (Dayton et al. (1999), Supra; Weiser et al. (1984) Acustica 56:114-119). Analysis of cell viability for each treatment type revealed no detectable adverse effects due to ultrasound and/or nanoparticles, indicating that enhancement occurs through contact-mediated mechanisms rather than through potentially destructive cavitational means.

1. A method for improved delivery of a therapeutic agent to a target,
comprising subjecting a nongaseous, lipid-encapsulated particle comprising a therapeutic agent with ultrasound energy at a frequency and mechanical index that enhances delivery of the agent to the target,
wherein the particle is located at the target, and
wherein the lipid encapsulating said particle is not disrupted during said delivery.

2. The method according to claim 1, wherein the particle comprises at least one fluorocarbon.

3. The method according to claim 2, wherein the fluorocarbon is perfluorooctane, perfluorooctylbromide, or perfluorodichlorooctane.

4. The method according to claim 1, wherein the targeting ligand is a polypeptide, a peptidomimetic, a polysaccharide, a lipid or a nucleic acid.

5. The method according to claim 4, wherein the polypeptide is at least a portion of an antibody.

6. The method according to claim 1, wherein the target resides in a mammalian subject.

7. The method according to claim 6, wherein the subject is human.

8. The method of claim 6, wherein said subject is diagnosed with a disease or condition and said therapeutic agent is selected as appropriate to said disease or condition.

9. The method of claim 1, wherein the particle is coupled to a targeting ligand.
